(19) United States
(12) Patent Application Publication
(10) Pub. No.: US 2010/0144779 A1
(43) Pub. Date: Jun. 10, 2010

(54) Marine Alkalod Makaluvamines and Derivatives Thereof

(75) Inventors: Sadanandan E. Velu, Birmingham, AL (US); Kevin P. Raisch, Trusville, AL (US); Ruiwen Zhang, Marietta, GA (US); Wei Wang, Birmingham, AL (US)

Correspondence Address: BRADLEY ARANT BOULT CUMMINGS LLP;INTELLECTUAL PROPERTY DEPARTMENT, 1819 FIFTH AVENUE NORTH, BIRMINGHAM, AL, 35203-2104, US

(73) Assignee: THE UAB RESEARCH FOUNDATION, Birmingham, AL (US)

(21) Appl. No.: 12/528,358

(22) PCT Filed: Feb. 25, 2008
(86) PCT No.: PCT/US08/02419

§ 371 Date: Jan. 27, 2010

Related U.S. Application Data
(60) Provisional application No. 60/891,250, filed on Feb. 23, 2007.

(51) Int. Cl. A61K 31/437; C07D 471/02; A61P 35/00

(52) U.S. Cl. 514/292; 546/84

(57) ABSTRACT

The present disclosure provides compounds based on the marine alkaloid makaluvamine. Described are compounds of the general formula (I) and (II). Also described are pharmaceutical compositions comprising one or more of the compounds of the general formula (I) and (II). The compounds and pharmaceutical compositions described inhibit the growth of several cancer lines, induce apoptosis and cell cycle arrest, display topoisomerase II inhibitory activity and modulate the activity and/or expression of key proteins involved in the regulation of cell growth. Methods of treatment and prevention using the compounds and pharmaceutical compositions described are also provided.

The present disclosure claims the benefit of U.S. Provisional Application No. 60/891,250, filed Feb. 23, 2007.

### STATEMENT AS TO FEDERAL FUNDING

The work described herein was supported by UAB Breast Spore Pilot Grant. As such, the Federal Government has certain rights in this invention.

### FIELD OF THE DISCLOSURE

The present disclosure relates generally to marine alkaloids. Specifically, the present disclosure relates to marine alkaloid makaluvamines and derivatives thereof. The present disclosure also relates to pharmaceutical compositions comprising the makaluvamines and derivatives thereof disclosed herein. Furthermore, the present disclosure relates to methods of using the compounds disclosed to modulate the activity of key proteins involved in the regulation of cell growth and to methods of treatment and prevention.

### BACKGROUND

For the past quarter of a century, global marine sources have proven to be a rich source of a vast array of new medicinally valuable compounds. These natural products exist as secondary metabolites in marine invertebrates such as sponges, bryazoa, tunicates and ascidians. As a result of the potential for new drug discovery, marine natural products have attracted scientists from different disciplines, such as organic chemistry, bioorganic chemistry, pharmacology and biology. About a dozen marine alkaloids are currently in various phases of human clinical trials for treatment of different cancers.

The largest numbers of bioactive marine alkaloids with novel structures have been isolated from marine sponges. Sponges produce a plethora of chemical compounds with widely varying carbon skeletons. Bioactive compounds from sponges have exhibited a variety of activities, such as anti-inflammatory, antitumor, immunosuppressive, neurosuppressive, antiviral, antimalarial and antibiotic activities. While a number of these alkaloids have been isolated in quantities sufficient to ascertain their biological profile, many with unique structures are available only in minute quantities, precluding their thorough biological evaluations. Laboratory synthesis of these alkaloids is the only practical solution to this problem.

Marine sponges of the genera Latrunculia, Batzella, Prianos and Zyzzya are a rich source of alkaloids bearing a pyrrolo[4,3,2-de]quinoline skeleton. This series of alkaloids comprise of about 60 metabolites including discorhabdins, epinardins, batzellines, isobatzellines, makaluvamines and veiutamine. Pyrrolo[4,3,2-de]quinoline alkaloids have shown a variety of biological activities such as inhibition of topoisomerase I and II, cytotoxicity against different tumor cell lines, antifungal and antimicrobial activities. Pyrrolo[4,3,2-de]quinoline alkaloids have recently received increasing attention as a source of new anticancer drugs. Their unique fused ring skeletons carrying interesting biological properties have made them targets for several synthetic and biological studies. There has been a rapid growth of interest in the synthesis and biological evaluation of this class of compounds and their analogs. Several reviews have been published on the chemistry and bioactivity of this class of compounds.

Makaluvamines A-P are a group of 16 marine alkaloids isolated mainly from four species of marine sponges, namely the Fijian sponge Zyzzya cf. marsailis, Indonesian sponge Histodermella sp, Pohnpeian sponge Zyzzya fuliginosa and Jamaican sponge Smenospongia aurea. Exemplary makaluvamine compounds known in the art are shown in FIG. 1.

Makaluvamines have exhibited in vitro cytotoxicity against the human colon tumor cell line, HCT-116. The cytotoxicities exhibited by makaluvamines against a Chinese hamster ovary (CHO) cell line Xrs-6 have paralleled the data obtained with HCT-116. The exact mechanism through which the makaluvamines exert their anticancer activity is not currently known, although the inhibition of DNA topoisomerase II has been postulated; however, makaluvamines may produce their anticancer activity by other mechanisms that are currently unknown.

Topoisomerases are vital nuclear enzymes which function to resolve topological dilemmas in DNA, such as overwinding, underwinding and catenation, which normally arise during replication, transcription and perhaps other DNA processes. These enzymes allow DNA to relax by forming enzyme-bridged strand breaks that act as transient gates or pivotal points for the passage of other DNA strands. Topoisomerase-targeting drugs appear to interfere with this breakage-reunion reaction of DNA topoisomerases. In the presence of topoisomerase inhibitors an aborted reaction intermediate, termed a ‘cleavable complex’, accumulates and results in replication/transcription arrest, which ultimately leads to cell death. The development of topoisomerase II inhibitors therefore offers an approach to the multi-regimental arsenal of therapies currently used in the clinic for the treatment of cancer.

Although many makaluvamines are known in the art, new derivatives are needed. The present disclosure provides a new range of makaluvamine derivatives not previously known in the art. The present disclosure shows that the disclosed makaluvamine derivatives are active against several cancer cell lines in vivo, modulate the activity of key proteins involved in the regulation of cell growth and are not toxic in in vivo studies. The present disclosure also provides methods of treatment and prevention utilizing the disclosed makaluvamine derivatives and methods of synthesizing the makaluvamine derivatives disclosed herein.

### BRIEF DESCRIPTION OF THE FIGURES

FIG. 1 shows the structure of makaluvamine A, makaluvamine D and makaluvamine F.

FIG. 2 shows an exemplary synthesis for the makaluvamine derivatives disclosed herein.

FIG. 3 shows the induction of apoptosis in various cancer cell lines after exposure to the makaluvamine derivatives disclosed herein. FIG. 3A shows the effect of 0, 0.1, 1.0, 10 and 25 μM concentrations of compound 4d, 28, 4c, 1 and 20 on induction of apoptosis in MCF-7 cells (upper panel) and MDA-468 cells (lower panel). FIG. 3B shows the effect of 0, 0.1, 1.0, 10 and 25 μM concentrations of compound 28, 4c and 1 on induction of apoptosis in A549 cells (upper panel) and H1299 cells (lower panel). FIG. 3C shows the effect of 0, 0.1, 1.0, 10 and 25 μM concentrations of compound 28 and 4c on induction of apoptosis in Panc-1 cells (upper panel) and IM R90-EEA cells (lower panel). FIG. 3D shows the effect of 0, 0.01, 0.1, 0.5 and 1 μM concentrations of compound 28 on induction of apoptosis in HCT-116 and HCT-116 (p53 minus) cells.

FIG. 4 shows the effect of the makaluvamine derivatives disclosed herein on expression of numerous proteins in human breast cancer cells. In FIG. 4A, MCF-7 cells were exposed to 1 μM concentration of the indicated compounds for 6 hours. Western blots were probed with antibodies specific for the proteins indicated. Numbers below each lane show densitometry readings for each lane as compared to control. In FIG. 4B, MCF-7 cells were exposed to the indicated concentration of compound 28 and incubated for 24 hours. Western blots were probed with antibodies specific for the proteins indicated.

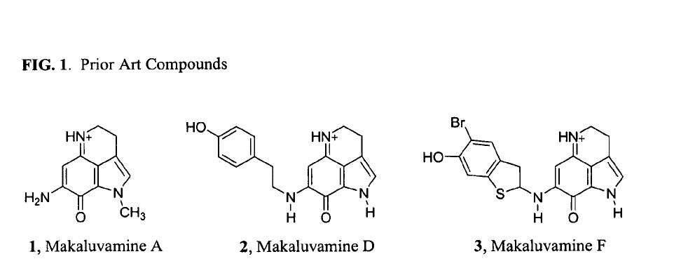

### DETAILED DESCRIPTION

The p53 tumor suppressor is a DNA damage-inducible sequence specific transcription factor and is activated through signaling pathways in response to stress. Depending on the conditions of cell growth, the type and duration of stress or DNA damage, p53 activates a different subset of target genes which can cause apoptosis, growth arrest, altered DNA repair, or altered differentiation. Among the multiple targets for the transcriptionally active p53 are cyclin-dependent kinase inhibitor p21Waf1, 14-3-3, reprimo, bax, DR5, p53AIP, PIDD, NOXA, PUMA, Fas/APO-1 and redox related genes. About 50% of human tumor types carry a p53 mutation. Most of the mutations are localized within the DNA binding domain, thereby affecting p53 transcriptional activities. Such mutations can partially or completely abrogate the ability of p53 to elicit transcriptional activities. As a result, the ability of p53 to elicit growth arrest, apoptosis, or both, is impaired and cell proliferation may continue unregulated.

MDM2, an ubiquitin ligase, is a cellular proto-oncogene that is over expressed in about 7% of all human cancers including sarcoma and cancers of the brain, lung, prostate and colon to name a few. Over-expression of MDM2 has been linked to a dire prognosis in many cancers including esophageal squamous cell carcinoma and prostate cancer as tumors in which MDM2 is over expressed tend to be more resistant to standard chemotherapy drugs. MDM2 is also correlated with increased metastasis in many cancers including breast and urothelial cancers. MDM2 contains a p53 binding domain, a nuclear localization signal, a central acidic domain and 3 zinc-finger motifs. The nuclear localization sequence allows MDM2 to constantly shuttle between the nucleus and the cytoplasm of the cell. MDM2 is now known to have p53-dependent activities and p53-independent activities as over expression of MDM2 in mice predisposes the mice to spontaneous tumor formation in the presence or absence of p53. The activity of MDM-2 has been recently described. See, e.g., Zhang and Zhang, 2005, Rayburn et al., 2005 and Zhang et al., 2005, each of which is incorporated by reference herein.

p53 and MDM2 regulate one another in a cyclic manner. MDM2 negatively regulates p53′ s activity by binding to p53 and keeping p53 functionally inactive. In fact, MDM2 is the principal cellular antagonist of p53, acting to limit the p53 proliferation-suppressive function in unstressed cells. MDM2 can inhibit p53 in multiple independent ways: by binding to its transcription activation domain, inhibiting p53 acetylation, promoting nuclear export, and by promoting proteasomal degradation of p53. After binding to p53, the MDM2 protein shuttles p53 protein out of the nucleus, into the cytoplasm where it is degraded. Consequently, over expression of MDM2 can lead to a loss of p53 activity which in turn leads to unregulated cell proliferation.

In turn, expression of MDM2 is controlled via a p53 responsive promoter. Inhibition of cell growth and marked cell death are often seen in the absence of p53 regulation by MDM2, further emphasizing the importance of the p53-MDM2 auto-regulatory loop in the control of cell growth and death.

MDM2 also has many p53 independent activities. For example, MDM2 is involved in normal muscle cell differentiation through the binding of transcription factor Sp1. Over expression of MDM2 has also been implicated with an increase in the pathogencity of HIV-1 and an increased risk for hemophilic synovitis I.

MDM2 also binds several other proteins including: pRb (tumor suppressor), E2F1/DP1 (transcription factor), MDM4 (a p53 binding protein), TGF-β1 (tumor suppressor), MTBP (involved in the regulation of the cell cycle), PML (tumor suppressor), p21Waf1/Cip1 (mediates p53-dependent cell cycle arrest), NPM (molecular chaperone), Merlin (involved in the regulation of cell growth and proliferation), PCAF (a co-activation protein), Tip 60 (histone acetyltransferase), several ribosomal proteins (including L5, L11 and L23), Numb (involved in the differentiation of neural cells), DNA polymerase E (involved in DNA repair), TSG101 (tumor susceptibility gene), YY1 (transcription factor), IGF-1R (insulin like growth factor), GR/ER (glucocorticoid receptor), AR (androgen receptor), HIF-1 (transcription factor), p73 (anti-tumor protein), p300 (transcription factor), NF-κB (involved in the regulation of apoptosis), PSD-95 (scaffolding protein) and others.

Therefore, MDM2 is a potential target for human therapeutics, including the treatment and prevention of cancer. Further, in light of its many p53-independent activities, compounds that target MDM2 may have therapeutic uses in addition to treating or preventing cancer such as inhibiting the pathogencity of HIV.

The present disclosure provides novel makaluvamine derivatives. The disclosed makaluvamine derivatives are active against several cancer cell lines in vivo, modulate the activity of key proteins involved in the regulation of cell growth, such as but not limited to, MDM2, p53 and topoisomerase II. The present disclosure also provides methods of treatment and prevention utilizing the disclosed makaluvamine derivatives. Methods of synthesizing the makaluvamine derivatives are disclosed herein.

Furthermore, the present disclosure provides new uses for the makaluvamine compounds and derivatives of the prior art. The ability of these compounds to modulate the activity of key signaling proteins involved in cell growth and proliferation was not appreciated in the art. The present disclosure shows for the first time the makaluvamine compounds and derivatives of the prior art are useful to modulate the activity of such key signaling proteins, such as, but not limited to, MDM2 and p53. The present disclosure also provides methods of treatment and prevention utilizing the makaluvamine compounds and derivatives of the prior art.

### DEFINITIONS

As used in this specification, the followings words and phrases have the meanings as defined below, unless otherwise limited in specific instances, either individually or as part of a larger group.

As used herein, the term “alkyl”, whether used alone or as part of a substituent group, includes straight hydrocarbon groups comprising from one to twenty carbon atoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃—, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)CH(CH₃)₂, —CH(CH₂CH₃)CH(CH₃)CH(CH₃)(CH₂CH₃), and others. The phrase also includes cyclic alkyl groups such as cyclopropyl, cyclobutyl, cyclopentyl, cyclohexyl, cycloheptyl, and cyclooctyl and such rings substituted with straight and branched chain alkyl groups as defined above. The phrase also includes polycyclic alkyl groups such as, but not limited to, adamantyl norbornyl, and bicyclo[2.2.2]octyl and such rings substituted with straight and branched chain alkyl groups as defined above.

As used herein, the term “alkenyl”, whether used alone or as part of a substituent group, includes an alkyl group having at least one double bond between any two adjacent carbon atoms.

As used herein, the term “alkynyl”, whether used alone or as part of a substituent group, includes an alkyl group having at least one triple bond between any two adjacent carbon atoms.

As used herein, the term “unsubstituted alkyl”, “unsubstituted alkenyl”, and “unsubstituted alkynyl” refers to alkyl, alkenyl and alkynyl groups that do not contain heteroatoms.

The phrase “substituted alkyl”, “substituted alkenyl”, and “substituted alkynyl” refers to alkyl, alkenyl and alkynyl groups as defined above in which one or more bonds to a carbon(s) or hydrogen(s) are replaced by a bond to non-hydrogen or non-carbon atoms such as, but not limited to, a halogen atom, such as F, Cl, Br, and I; and oxygen atom in groups such as carbonyl, carboxyl, hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, enamines imines, oximes, hydrazones, and nitriles; a silicon atom in groups such as in trialkylsilyl groups, dialkylarylsilyl groups, alkyldiarylsilyl groups, and triarylsilyl groups; and other heteroatoms in various other groups. Other alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an amine, alkylamine, dialkylamine, arylamine, (alkyl)(aryl)amine, diarylamine, heterocyclylamine, (alkyl)(heterocyclyl)amine, (aryl)(heterocyclyl)amine, or diheterocyclylamine group.

As used herein, the term “unsubstituted aryl” refers to monocyclic or bicyclic aromatic hydrocarbon groups having 6 to 12 carbon atoms in the ring portion, such as, but not limited to, phenyl, naphthyl, anthracenyl, biphenyl and diphenyl groups, that do not contain heteroatoms. Although the phrase “unsubstituted aryl” includes groups containing condensed rings such as naphthalene, it does not include aryl groups that have other groups such as alkyl or halogen groups bonded to one of the ring members, as aryl groups such as tolyl are considered herein to be substituted aryl groups as described below. Unsubstituted aryl groups may be bonded to one or more carbon atom(s), oxygen atom(s), nitrogen atom(s), and/or sulfur atom(s) in the parent compound, however.

As used herein, the term “substituted aryl group” has the same meaning with respect to unsubstituted aryl groups that substituted alkyl groups had with respect to unsubstituted alkyl groups. However, a substituted aryl group also includes aryl groups in which one of the aromatic carbons is bonded to one of the non-carbon or non-hydrogen atoms described above and also includes aryl groups in which one or more aromatic carbons of the aryl group is bonded to a substituted and/or unsubstituted alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the phrase “substituted aryl” includes, but is not limited to tolyl, and hydroxyphenyl among others. Substituted aryl groups may be bonded to one or more carbon atom(s), oxygen atom(s), nitrogen atom(s), and/or sulfur atom(s) in the parent compound, however.

As used herein, the term “unsubstituted aralkyl” refers to unsubstituted alkyl, alkenyl or alkyl groups as defined above in which a hydrogen or carbon bond of the unsubstituted alkyl, alkenyl or alkyl group is replaced with a bond to an unsubstituted or substituted aryl group as defined above. For example, methyl (CH₃) is an unsubstituted alkyl group. If a hydrogen atom of the methyl group is replaced by a bond to a phenyl group, such as if the carbon of the methyl were bonded to a carbon of benzene, then the compound is an unsubstituted aralkyl group (i.e., a benzyl group). Unsubstituted aralkyl groups may be bonded to one or more carbon atom(s), oxygen atom(s), nitrogen atom(s), and/or sulfur atom(s) in the parent compound, however.

As used herein, the term “substituted aralkyl” has the same meaning with respect to unsubstituted aralkyl groups that substituted aryl groups had with respect to unsubstituted aryl groups. However, a substituted aralkyl group also includes groups in which a carbon or hydrogen bond of the alkyl, alkenyl or alkyl part of the group is replaced by a bond to a non-carbon or a non-hydrogen atom.

As used herein, the term “unsubstituted heterocyclyl” refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to, N, O, and S. Although the phrase “unsubstituted heterocyclyl” includes condensed heterocyclic rings such as benzimidazolyl, it does not include heterocyclyl groups that have other groups such as alkyl or halogen groups bonded to one of the ring members, as compounds such as 2-methylbenzimidazolyl are “substituted heterocyclyl” groups as defined below. Examples of heterocyclyl groups include, but are not limited to: unsaturated 3 to 8 membered rings containing 1 to 4 nitrogen atoms such as, but not limited to pyrrolyl, pyrrolinyl, imidazolyl, pyrazolyl, pyridyl, dihydropyridyl, pyrimidyl, pyrazinyl, pyridazinyl, triazolyl, tetrazolyl; saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms and 1 to 3 nitrogen atoms such as, but not limited to, oxazolyl, isoxazolyl, oxadiazolyl; saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4-dihydrobenzothiazinyl, etc.), unsaturated 3 to 8 membered rings containing oxygen atoms such as, but not limited to furyl; unsaturated condensed heterocyclic rings containing 1 to 2 oxygen atoms such as benzodioxolyl (e.g. 1,3-benzodioxoyl, etc.); unsaturated 3 to 8 membered rings containing an oxygen atom and 1 to 2 sulfur atoms such as, but not limited to, dihydrooxathiinyl; saturated 3 to 8 membered rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene, tetrahydrothiophene oxide, and tetrahydrothiophene 1,1-dioxide. Preferred heterocyclyl groups contain 5 or 6 ring members. More preferred heterocyclyl groups include morpholine, piperazine, piperidine, pyrrolidine, imidazole, pyrazole, 1,2,3-triazole, 1,2,4-triazole, tetrazole, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran.

As used herein, the term “substituted heterocyclyl” refers to an unsubstituted heterocyclyl group as defined above in which one of the ring members is bonded to a non-hydrogen atom such as described above with respect to substituted alkyl groups and substituted aryl groups. Examples, include, but are not limited to, 2-methylbenzimidazolyl, 5-methylbenzimidazolyl, 5-chlorobenzthiazolyl, 1-methyl piperazinyl, and 2-chloropyridyl among others.

The phrase “unsubstituted heterocyclylalkyl” refers to unsubstituted alkyl, alkenyl or alkynyl groups as defined above in which a hydrogen or carbon bond of the unsubstituted alkyl, alkenyl or alkynyl group is replaced with a bond to a substituted or unsubstituted heterocyclyl group as defined above. For example, methyl (—CH₃) is an unsubstituted alkyl group. If a hydrogen atom of the methyl group is replaced by a bond to a substituted or unsubstituted heterocyclyl group, such as if the carbon of the methyl were bonded to carbon 2 of pyridine (one of the carbons bonded to the N of the pyridine) or carbons 3 or 4 of the pyridine, then the compound is an unsubstituted heterocyclylalkyl group.

The phrase “substituted heterocyclylalkyl” refers to substituted alkyl, alkenyl or alkynyl groups as defined above in which a hydrogen or carbon bond of the substituted alkyl, alkenyl or alkynyl group is replaced with a bond to a substituted or unsubstituted heterocyclyl group as defined above. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group.

The phrase “halogen” or “halo” refers to fluorine, chlorine, bromine and iodine.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “prodrug” is meant to include functional derivatives of the compounds disclosed which are readily convertible in vivo into the required compound. Thus, in the methods of treatment of the present disclosure, the term “administering” shall encompass the treatment of the various disease states/conditions described with the compound specifically disclosed or with a prodrug which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The terms “prevent”, “preventing”, “prevention” “suppress”, “suppressing” and suppression as used herein refer to administering a compound prior to the onset of clinical symptoms of a disease state/condition so as to prevent any symptom, aspect or characteristic of the disease state/condition. Such preventing and suppressing need not be absolute to be useful.

The terms “treat”, “treating” and “treatment” as used herein refers to administering a compound after the onset of clinical symptoms of a disease state/condition so as to reduce or eliminate any symptom, aspect or characteristic of the disease state/condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, and may include the knowledge that the patient is ill as the result of a disease state/condition that is treatable by a compound or pharmaceutical composition of the disclosure.

The term “in need of prevention” as used herein refers to a judgment made by a caregiver that a patient requires or will benefit from prevention. This judgment is made based on a variety of factors that are in the realm of a caregiver's expertise, and may include the knowledge that the patient may become ill as the result of a disease state/condition that is treatable by a compound or pharmaceutical composition of the disclosure.

The term “individual” or “patient” as used herein refers to any animal, including mammals, such as, but not limited to, mice, rats, other rodents, rabbits, dogs, cats, swine, cattle, sheep, horses, or primates, or humans. The term may specify male or female or both, or exclude male or female.

The term “therapeutically effective amount”, in reference to the treating, preventing or suppressing of a disease state/condition, refers to an amount of a compound either alone or as contained in a pharmaceutical composition that is capable of having any detectable, positive effect on any symptom, aspect, or characteristics of the disease state/condition. In one embodiment, a therapeutically effective amount is a tumor growth inhibiting amount. Such effect need not be absolute to be beneficial.

### Methods of Treatment and Prevention

The present disclosure provides compounds based on the marine alkaloid makaluvamine. In one embodiment of the present disclosure, compounds based on the marine alkaloid makaluvamine of the general formula I and II are provided.

The present disclosure describes the use of the disclosed compounds to prevent or treat cancer and diseases caused or related to bacterial infections. The present disclosure also provides for methods to treat and/or prevent diseases or conditions characterized by and/or unregulated cellular proliferation. Further, the present disclosure provides for methods to treat and/or prevent diseases or conditions treatable or preventable by inhibiting or decreasing topoisomerase II activity, inhibiting or decreasing MDM2 activity or increasing/restoring p53 activity, or modulating (increasing or decreasing) expression of MDM2, E2F1, PARP, cdc2, cdc25c, p53 and/or p21. Still further, the present disclosure provides for methods to treat and/or prevent diseases or conditions by causing cell cycle arrest, including without limitation, cell cycle arrest in the S and G2/M phases, or inducing apoptosis.

In one embodiment, the teachings of the present disclosure provide for treating and/or preventing cancer in a subject or a disease in a subject in need of such treatment or prevention. The method of treatment comprises the steps of identifying a subject in need of such treatment or prevention and administering at least one the marine alkaloid makaluvamine of the general formula I and II, or a pharmaceutically acceptable salt thereof, to the subject. In a specific embodiment, the marine alkaloid makaluvamine of the general formula I and II is administered in a therapeutically effective amount. The marine alkaloid makaluvamine of the general formula I and II may be administered alone or as a part of a pharmaceutical composition or medicament. In one embodiment, the compounds are any one or more of compounds 4a-4-g, 7c-7g or compounds 1-30; in an alternate embodiment, the compounds are compounds 4d-4-g, 7c-7g, 1-9, 11-13, 15-17, 19-21, 23-27, and/or 29-30. Such administration of the marine alkaloid makaluvamine of the general formula I and II would thereby treat/prevent such cancer. In one embodiment, the administration of marine alkaloid makaluvamine of the general formula I and II modulates the activity of topoisomerase II, MDM2, E2F1, Cdc2, cdc25c, p21 and/or p53; in a specific embodiment, the compounds decrease the activity, at least in part, of topoisomerase II, MDM2, E2F1, Cdc2, cdc25c and/or p21 and/or increase/restore the activity, at least in part, of p53. In another embodiment, the administration of marine alkaloid makaluvamine of the general formula I and II modulates the expression of MDM2, E2F1, Cdc2, cdc25c, p21 and/or p53; in a specific embodiment, the compounds decrease the expression, at least in part, of MDM2, E2F1, Cdc2, cdc25c and/or p21 and/or increase/restore the expression at least in part, of p53 and/or p21. In still a further embodiment, the administration of marine alkaloid makaluvamine of the general formula I and II increases apoptosis. In a further alternate embodiment, the administration of marine alkaloid makaluvamine of the general formula I and II causes cell cycle arrest, including without limitation, cell cycle arrest in the S and G2/M phases.

In another embodiment, the teachings of the present disclosure provide for treating and/or preventing a condition characterized by unregulated cellular proliferation in a subject in need of such treatment or prevention. The method of treatment comprises the steps of identifying a subject in need of such treatment or prevention and administering at least one the marine alkaloid makaluvamine of the general formula I and II, or a pharmaceutically acceptable salt thereof, to the subject. In a specific embodiment, the marine alkaloid makaluvamine of the general formula I and II is administered in a therapeutically effective amount. The marine alkaloid makaluvamine of the general formula I and II may be administered alone or as a part of a pharmaceutical composition or medicament. In one embodiment, the compounds are any one or more of compounds 4a-4-g, 7c-7g or compounds 1-30; in an alternate embodiment, the compounds are compounds 4d-4-g, 7c-7g, 1-9, 11-13, 15-17, 19-21, 23-27, and/or 29-30. Such administration of the marine alkaloid makaluvamine of the general formula I and II would thereby treat/prevent the disease or condition characterized by unregulated cellular proliferation. In one embodiment, the administration of marine alkaloid makaluvamine of the general formula I and II inhibits modulates the activity of topoisomerase II, MDM2, E2F1, Cdc2, cdc25c, p21 and/or p53; in a specific embodiment, the compounds decrease the activity, at least in part, of topoisomerase II, MDM2, E2F1, Cdc2, cdc25c and/or p21 and/or increase/restore the activity, at least in part, of p53. In another embodiment, the administration of marine alkaloid makaluvamine of the general formula I and II modulates the expression of MDM2, E2F1, Cdc2, cdc25c, p21 and/or p53; in a specific embodiment, the compounds decrease the expression, at least in part, of MDM2, E2F1, Cdc2, cdc25c and/or p21 and/or increase/restore the expression at least in part, of p53 and/or p21. In still a further embodiment, the administration of marine alkaloid makaluvamine of the general formula I and II increases apoptosis. In a further alternate embodiment, the administration of marine alkaloid makaluvamine of the general formula I and II causes cell cycle arrest, including without limitation, cell cycle arrest in the S and G2/M phases.

In an alternate embodiment, the teachings of the present disclosure provide for the prevention or treatment of a disease or condition characterized by decreased p53 activity and/or expression in a subject in a subject in need of such treatment or prevention. The method of prevention or treatment comprises the steps of identifying a subject in need of such treatment or prevention and administering at least one the marine alkaloid makaluvamine of the general formula I and II, or a pharmaceutically acceptable salt thereof, to said subject. In a specific embodiment, the marine alkaloid makaluvamine of the general formula I and II is administered in a therapeutically effective amount. The marine alkaloid makaluvamine of the general formula I and II may be administered alone or as a part of a pharmaceutical composition or medicament. Such administration of the marine alkaloid makaluvamine of the general formula I and II would thereby treat/prevent the disease or condition characterized by decreased p53 activity and/or expression by increasing or restoring, at least in part, p53 activity and/or expression. In one embodiment, p53 activity is increased or restored via inhibition of MDM2 activity. In one embodiment, the compounds are any one or more of compounds 4a-4-g, 7c-7g or compounds 1-30; in an alternate embodiment, the compounds are compounds 4c, 4, 2, 5, 1, 12, 24, 17, 29, 30, 26, 4d, 4f and/or 7c. In one case, the administration of marine alkaloid makaluvamine of the general formula I and II increases apoptosis. In a further case, the administration of marine alkaloid makaluvamine of the general formula I and II causes cell cycle arrest, including without limitation, cell cycle arrest in the S and G2/M phases.

In a further alternate embodiment, the teachings of the present disclosure provide for the prevention or treatment of a disease or condition characterized by increased MDM2 activity and/or expression in a subject in a subject in need of such treatment or prevention. The method of prevention or treatment comprises the steps of identifying a subject in need of such prevention and administering at least one the marine alkaloid makaluvamine of the general formula I and II, or a pharmaceutically acceptable derivative thereof, to said subject. In a specific embodiment, the marine alkaloid makaluvamine of the general formula I and II is administered in a therapeutically effective amount. The marine alkaloid makaluvamine of the general formula I and II may be administered alone or as a part of a pharmaceutical composition or medicament. Such administration would thereby treat or prevent the disease or condition characterized by increased MDM2 activity and/or expression by decreasing, at least in part, MDM2 activity and/or expression. In one embodiment, the compounds are any one or more of compounds 4a-4-g, 7c-7g or compounds 1-30; in an alternate embodiment, the compounds are compounds 4c, 28, 4, 2, 8, 4e, 12, 15, 16, 20, 21, 24 and/or 7c. In one case, the administration of marine alkaloid makaluvamine of the general formula I and II increases apoptosis. In a further case, the administration of marine alkaloid makaluvamine of the general formula I and II causes cell cycle arrest, including without limitation, cell cycle arrest in the S and G2/M phases.

In a further alternate embodiment, the teachings of the present disclosure provide for the prevention or treatment of a disease or condition characterized by increased activity and/or expression of p21, E2F1, cdc2 and/or cdc25c in a subject in a subject in need of such treatment/prevention. The method of prevention or treatment comprises the steps of identifying a subject in need of such treatment/prevention and administering at least one the marine alkaloid makaluvamine of the general formula I and II, or a pharmaceutically acceptable salt thereof, to said subject. In a specific embodiment, the marine alkaloid makaluvamine of the general formula I and II is administered in a therapeutically effective amount. The marine alkaloid makaluvamine of the general formula I and II may be administered alone or as a part of a pharmaceutical composition or medicament. Such administration would thereby prevent the disease or condition by decreasing, at least in part, the activity and/or expression of MDM2, E2F1, PARP, cdc2 and/or cdc25c. In one embodiment, the compounds are any one or more of compounds 4a-4-g, 7c-7g or compounds 1-30. In one case, the administration of marine alkaloid makaluvamine of the general formula I and II increases apoptosis. In a further case, the administration of marine alkaloid makaluvamine of the general formula I and II causes cell cycle arrest, including without limitation, cell cycle arrest in the S and G2/M phases.

In an alternate embodiment, the teachings of the present disclosure provide for the prevention or treatment of a disease or condition characterized by an increase in topoisomerase II activity in a subject in need of such treatment or prevention. The method of prevention or treatment comprises the steps of identifying a subject in need of such treatment/prevention and administering at least one the marine alkaloid makaluvamine of the general formula I and II, or a pharmaceutically acceptable salt thereof, to said subject. In a specific embodiment, the marine alkaloid makaluvamine of the general formula I and II is administered in a therapeutically effective amount. The marine alkaloid makaluvamine of the general formula I and II may be administered alone or as a part of a pharmaceutical composition or medicament. Such administration would thereby prevent the disease or condition characterized by an increase in topoisomerase II activity by decreasing, at least in part, the activity of topoisomerase II. In one embodiment, the compounds are any one or more of compounds 4a-4-g, 7c-7g or compounds 1-30. In one case, the administration of marine alkaloid makaluvamine of the general formula I and II increases apoptosis. In a further case, the administration of marine alkaloid makaluvamine of the general formula I and II causes cell cycle arrest, including without limitation, cell cycle arrest in the S and G2/M phases.

In the above methods of treatment and prevention, such diseases or conditions include, but are not limited to, leukemias and lymphomas such as acute lymphocytic leukemia, acute nonlymphocytic leukemias, chronic lymphocytic leukemia, chronic myelogenous leukemia, Hodgkin's Disease, non-Hodgkin's lymphomas, and multiple myeloma, childhood solid tumors such as brain tumors, neuroblastoma, retinoblastoma, Wilms Tumor, bone tumors, and soft-tissue sarcomas, common solid tumors of adults such as lung cancer, colon cancer, rectal cancer, breast cancer, prostate cancer, urinary cancers, uterine cancers, oral cancers, pancreatic cancer, melanoma and other skin cancers, stomach cancer, ovarian cancer, brain tumors, liver cancer, laryngeal cancer, thyroid cancer, esophageal cancer, and testicular cancer. In a specific embodiment, in the above methods of treatment and prevention, such diseases or conditions include, but are not limited to, leukemia, colon, melanoma, ovarian, renal, prostate, lung and breast cancer as well as cancers of the central nervous system.

In still a further alternate embodiment, the teachings of the present disclosure provide for the prevention or treatment of a disease or condition caused by or related to a bacterial infection, including both gram-positive and gram negative bacterial infections. The method of prevention or treatment comprises the steps of identifying a subject in need of such treatment/prevention and administering at least one the marine alkaloid makaluvamine of the general formula I and II, or a pharmaceutically acceptable salt thereof, to said subject. In a specific embodiment, the marine alkaloid makaluvamine of the general formula I and II is administered in a therapeutically effective amount. The marine alkaloid makaluvamine of the general formula I and II may be administered alone or as a part of a pharmaceutical composition or medicament. Such administration would thereby prevent the disease or condition caused by or related to a bacterial infection. Exemplary bacteria that may cause a human disease state or condition that may be treated by the compounds and pharmaceutical compositions disclosed herein include, but are not limited to, Legionella species, Campylobacter species, Staphylococcus species, E. coli species, Borrelia species, Helicobacter species, Ehrlichia species, Clostridium species, Vibrio species, Bartonella species, Streptococcus species, Chlamydia species, Clostridium species, Neisseria species, Pseudomonas species, Xanthomonas species, Agrobacterium species, Brucella species, Francisella species, Vibrio species, Acinetobacter species, Haemophilus species, Salmonella species, Yersinia species, Bacillus species, Corynebacterium species, Mycobacterium, species, Chlamydia species, Mycoplasma species, Klebsiella species, Salmonella species, Leptospirosis species, Fusobacterium species, Listeria species, Proteus species, Bacteroides species, and Porphyromonas species. The method of prevention or treatment comprises the steps of identifying a subject in need of such treatment/prevention and administering at least one the marine alkaloid makaluvamine of the general formula I and II, or a pharmaceutically acceptable salt thereof, to said subject. In a specific embodiment, the marine alkaloid makaluvamine of the general formula I and II is administered in a therapeutically effective amount. The marine alkaloid makaluvamine of the general formula I and II may be administered alone or as a part of a pharmaceutical composition or medicament. Such administration would thereby prevent the disease or condition caused by or related to a bacterial infection.

The methods of the treating and preventing herein also comprises further administering of a chemotherapeutic agent in combination with and of the compounds or pharmaceutical compositions of the present disclosure. Any suitable chemotherapeutic agent can be employed for this purpose. The chemotherapeutic agent is typically selected from the group consisting of alkylating agents, antimetabolites, natural products, hormonal agents, and miscellaneous agents.

Examples of alkylating chemotherapeutic agents include carmustine, chlorambucil, cisplatin, lomustine, cyclophosphamide, melphalan, mechlorethamine, procarbazine, thiotepa, uracil mustard, triethylenemelamine, busulfan, pipobroman, streptozocin, ifosfamide, dacarbazine, carboplatin, and hexamethylmelamine.

Examples of chemotherapeutic agents that are antimetabolites include cytosine arabinoside, fluorouracil, gemcitabine, hydroxyurea, mercaptopurine, methotrexate, azaserine, thioguanine, floxuridine, fludarabine, cladribine and L-asparaginase.

Examples of chemotherapeutic agents that are natural products include actinomycin D, bleomycin, camptothecins, daunomycin, doxorubicin, etoposide, mitomycin C, TAXOL (paclitaxel), taxotere, teniposide, vincristine, vinorelbine, mithramycin, idarubicin, MITHRACIN™ (plicamycin), and deoxycoformycin. An example of a hormonal chemotherapeutic agent includes tamoxifen. Examples of the aforesaid miscellaneous chemotherapeutic agents include mitotane, mitoxantrone, vinblastine, and levamisole.

Other embodiments and aspects of the invention will be apparent according to the description provided below.

### Makaluvamine Derivatives

This present disclosure provides compounds of the general formula (I) and (II), or pharmaceutically acceptable salts thereof, or esters thereof, or prodrugs thereof and tautomers and polymorphic variants of any of the foregoing.

With regard to compounds having the general formula (I), the compounds are

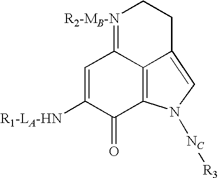

### Wherein

R₁ is selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;

R₂ is H or is selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;

R₃ is H or is selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;

L, M and N are each an optional linker which are independently selected from a substituted or unsubstituted alkyl, substituted or unsubstituted alkenyl, substituted or unsubstituted alkynyl;

A, B and C are independently selected from 1 or 0; when R₂ and R₃ are H, then B and C are each equal to 0 and when R₁═CH₃, A=0.

In one embodiment, R₁ is selected from substituted or unsubstituted alkyl, substituted or unsubstituted aralkyl, or substituted or unsubstituted heterocyclylalkyl and R₂ and R₃ are independently selected from H, substituted or unsubstituted alkyl, substituted or unsubstituted alkenyl, substituted or unsubstituted alkynyl.

In another embodiment, R₁ is selected from substituted or unsubstituted alkyl, substituted or unsubstituted aralkyl, or substituted or unsubstituted heterocyclylalkyl and wherein R₂ and R₃ are each equal to H and B and C are each =to 0.

In a further embodiment, L, M and N are each independently selected from a chain of 2-15 carbon atoms selected from a substituted or unsubstituted alkyl, substituted or unsubstituted alkenyl, substituted or unsubstituted alkynyl. In a yet a further specific embodiment, the L, M and N are each independently selected from a chain of 2-3 carbon atoms selected from a substituted or unsubstituted alkyl, substituted or unsubstituted alkenyl, substituted or unsubstituted alkynyl.

In yet a further embodiment, R₁ is selected from the substituents groups illustrated in Tables 1 and 2, wherein R₂ and R₃ are each equal to H and B and C are each =to 0.

In still a further embodiment, when R₁ is selected from straight chain alkyl of 1-10 carbon atoms, furan, thiopene, napthyl, pyridyl, phenyl, mono-substituted phenyl or di-substituted phenyl (where the substitutions include the following: a straight chain alkyl of 1-4 carbon atoms, trifluoromethyl, nitro, an O-alkyl of 1-4 carbon atoms, hydroxyl, F, Cl, Br, NRₓRₓ, where Rₓ is a straight chain alkyl from 1-4 carbon atoms, or CO₂Rₓ),

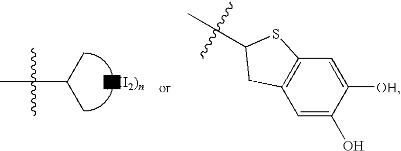

L is not CH₂ and when L is a substituted or unsubstituted alkyl or alkenyl of 2 carbon atoms in length, the R₁ is not

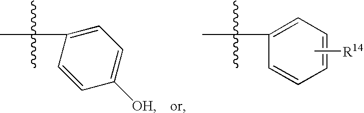

Where R₁₄ is F, Cl, Br, NO₂, straight chain alkyl of 1 to 4 carbons, O-alkyl of 1 to 4 carbons or trifluoromethyl.

With regard to compounds having the general formula (II),

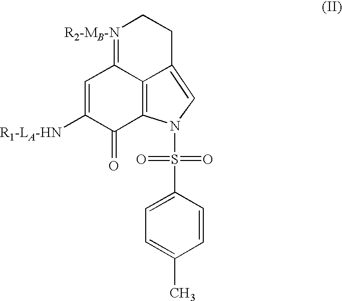

### Wherein

R₁ is selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;

R₂ is H or is selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;

L and M are each an optional linker which are independently selected from a substituted or unsubstituted alkyl, substituted or unsubstituted alkenyl, substituted or unsubstituted alkynyl;

A is 1; and

A and B are each independently selected from 1 or 0; when R₂ is H, then B is equal to 0 and when R₁═CH₃, A=0.

In one embodiment, R₁ is selected from substituted or unsubstituted alkyl, substituted or unsubstituted aralkyl, or substituted or unsubstituted heterocyclylalkyl and R₂ is selected from H, substituted or unsubstituted alkyl, substituted or unsubstituted alkenyl, substituted or unsubstituted alkynyl.

In another embodiment, R₁ is selected from substituted or unsubstituted alkyl, substituted or unsubstituted aralkyl, or substituted or unsubstituted heterocyclylalkyl and wherein R₂ is equal to H and B is =to 0.

In a further embodiment, L and M are each independently selected from a chain of 2-15 carbon atoms selected from a substituted or unsubstituted alkyl, substituted or unsubstituted alkenyl, substituted or unsubstituted alkynyl. In a yet a further specific embodiment, L and M are each independently selected from a chain of 2-3 carbon atoms selected from a substituted or unsubstituted alkyl, substituted or unsubstituted alkenyl, substituted or unsubstituted alkynyl.

In yet a further embodiment, R₁ is selected from the substituents groups illustrated in Tables 1 and 2, wherein R₂ is equal to H and B is =to 0.

### Exemplary Synthesis

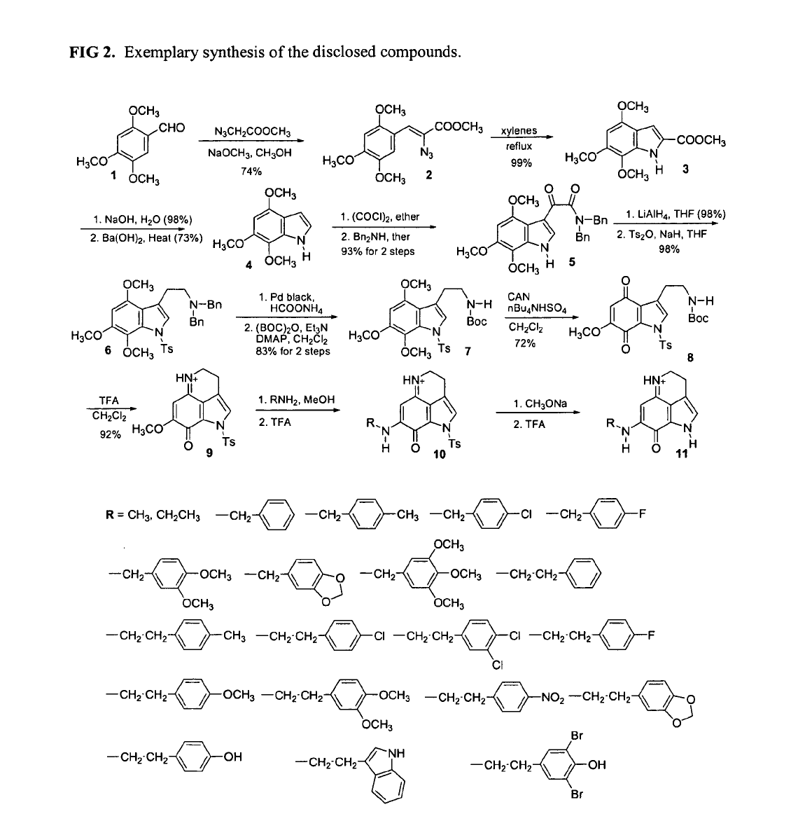

The makaluvamine compounds were prepared by introducing a variety of substitutions at the 7-position of the pyrroloiminoquinone ring present in makaluvamines. An exemplary synthesis is shown in FIG. 2. Referring to FIG. 2, commercially available 2,4,5-trimethoxybenzaldehyde 1 was converted to 4,6,7-trimethoxyindole 4 in four steps. Following the general Rees-Moody protocol, the aldehyde 1 was condensed with methyl azidoacetate in the presence of sodium methoxide in methanol to form the azidocinnamate 2, giving a 74% yield. Thermolysis of compound 2 in refluxing xylenes afforded 4,6,7-trimethoxyindole-2-carboxylate 3 in a 99% yield. Alkaline hydrolysis of compound 3 using sodium hydroxide followed by the decarboxylation of the acid by heating with barium hydroxide led to the production of 4,6,7-trimethoxyindole 4 with a 73% yield. Reaction of compound 4 with oxalyl chloride gave the 3-glyoxalyl chloride, which upon treatment with dibenzylamine in ether, produced the glyoxamide derivative 5 (93% yield). Reduction of glyoxamide 5 with lithium aluminum hydride afforded the corresponding N,N-dibenzyltryptamine derivative (98% yield), which was converted to the corresponding N-tosyl derivative 6 (98% yield) by treatment with tosic anhydride in the presence of sodium hydride in N,N-dimethylformamide (DMF). Debenzylation of compound 6 was carried out by transfer hydrogenolysis using a mixture of ammonium formate/fomic acid in the presence of palladium black to produce a tryptamine, which was directly converted to N-Boc protected amine 7 by treatment with (Boc)₂O in the presence of triethyl amine and N,N-dimethylaminopyridine (83% yield for 2 steps). N-Boc protected amine 7 was converted to the corresponding quinone 8 (72% yield) by oxidation using ceric ammonium nitrate (CAN) in the presence of tetrabutyl ammonium hydrogen sulfate as a phase transfer catalyst in a dichloromethane/water solvent system. The quinone 8 was readily converted to the pyrroloiminoquinone salt 9 (92% yield) by treatment with trifluoroacetic acid. Treatment of compound 9 with various amines in methanol afforded the aminated products 10. Detosylation of compound 10 using sodium methoxide in methanol afforded the final products 11.

Additional methods of synthesis for the compounds disclosed herein can be found in Shinkre et al. 2007 (Analogs of the marine alkaloid makaluvamines: Synthesis, topoisomerase II inhibition, and anticancer activity, Bioorganic and Medicinal Chemistry Letters, Vol. 17, No. 10, pp 2890-2893, 2007) and Shinkre et al. 2008 (Synthesis and antiproliferative activity of benzyl and phenethyl analogs of makaluvamines, Bioorganic and Medicinal Chemistry, accepted Nov. 20, 2007, In Press, 2008). Each of the foregoing are hereby incorporated by reference herein in their entirety.

### Inhibition of Cancer Cell Growth

The makaluvamine compounds described herein were evaluated for their ability to inhibit the growth of a number of different cancer cell lines in vitro.

In initial experiments, compounds 4a-g and 7c-g were evaluated for their cytotoxicity against human breast cancer cell lines MCF-7 and MDA-MB-468 and human colon cancer cell line HCT-116. The makaluvamine compounds provided in Table 2 were also evaluated for their cytotoxicity against human breast cancer cell lines MCF-7. Compound nos. 8 and 27-33 were not evaluated in this experiment. Cells were obtained from ATCC (Manassas, Va.). For each derivative the IC₅₀ value was determined using data generated from 2-4 independent tetrazolium-based (XTT) cytotoxicity assays (R & D Systems Inc., Minneapolis, Minn.) according to the manufacturer's instructions. The IC₅₀ is defined as the dose of the compound that inhibits 50% cell proliferation. MCF-7, MDA-MD-468 and HCT-116 cells were grown at 37 degrees C. in a humidified atmosphere with complete growth media supplemented with fetal bovine serum. Growth media was changed 2-3 times per week as required.

Two known topoisomerase II targeting drugs, m-AMSA and etoposide, were included for comparisons. Results of cytotoxic assays are summarized in Table 1 for the makaluvamine derivatives 4a-g and 7c-g and in Table 2 for the makaluvamine derivatives identified as compound nos. 1-4 and 6-23. With regard to the results in Table 1, HCT-116 cells were shown to be the most sensitive to etoposide and m-AMSA with IC₅₀ doses of 1.7 μM and 0.7 μM, respectively. MDA-MB-468 cells showed IC₅₀ values of 13.6 μM and 8.5 μM for etoposide and m-AMSA, respectively. MCF-7 cells were shown to be the least sensitive with IC₅₀ values of 35.6 μM and 21.7 μM for etoposide and m-AMSA, respectively. Several of makaluvamine analogs inhibited cell growth at least as effectively as or better than the control drugs in these assays. Four makaluvamine analogs (4c, 7d, 7f and 7g) exhibited lower IC₅₀ values against HCT-116 as compared to control drug etoposide. One analog (7d) exhibited lower IC₅₀ value against HCT-116 as compared to m-AMSA. All twelve of the makaluvamine analogs exhibited lower IC₅₀ values against MCF-7 and MDA-MB-468 as compared to etoposide as well as m-AMSA. The makaluvamines derivative that showed the best activity against HCT-116 was the N-tosyl-6-phenethylamino derivative (7d) with an IC₅₀ value of 0.5 μM. The compound that showed best activity against MCF-7 is the benzyl amino derivative (4c) with an IC₅₀ value of 1.0 μM. Benzyl amino derivative, 4c and phenethyl amino derivative, 4d showed best activity against MDA-MB-468 with IC₅₀ value of 0.3 μM for each.

With regard to the results of Table 2, all of the makaluvamine derivatives inhibited cell growth better than etoposide in MCF-7 cells, with the exception of compound no. 4 (IC₅₀ value of 30.5 μM), which equally as effective towards inhibiting MCF-7 cell growth as etoposide (IC₅₀ value of 30.5 μM). Compound nos. 1, 3, 7, 17, 19 and 21 inhibited MCF-7 cell growth with IC₅₀ values of less than 1 μM (range of 0.65 to 0.96 μM). Compound nos. 6, 8-16 and 22-23 inhibited MCF-7 cell growth with IC₅₀ values ranging from 1 to 5 μM, while compound nos. 2, 18 and 20 inhibited MCF-7 cell growth with IC₅₀ values from 5-15 μM.

Additionally, as shown in Table 3, several of the disclosed compounds were tested against the cell panel of the National Cancer Institute Developmental Therapeutics Program. This panel implements the NCI60 cell line screen, including leukemia, non-small cell lung cancer, colon cancer, central nervous system cancer, melanoma, ovarian cancer, renal cancer, prostate cancer and breast cancer. The cell lines tested are specified in Table 3.

Generally, the human tumor cell lines of the cancer screening panel are grown in RPMI 1640 medium containing 5% fetal bovine serum and 2 mM L-glutamine. For a typical screening experiment, cells are inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO₂, 95% air and 100% relative humidity for 24 h prior to addition of experimental drugs. After 24 h, two plates of each cell line are fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or ½ log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 h at 37° C., 5% CO₂, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements (time zero, Tz, control growth, (control), and test growth in the presence of drug at the five concentration levels (Ti)), the percentage growth is calculated at each of the drug concentrations levels. Percentage growth inhibition is calculated as:

[(Ti−Tz)/(C−Tz)]×100 for concentrations for which Ti>/=Tz

[(Ti−Tz)/Tz]×100 for concentrations for which Ti<Tz.

Growth inhibition of 50% (GI₅₀) is calculated from [(Ti−Tz)/(C−Tz)]×100=50, which is the drug concentration resulting in a 50% reduction in the net protein increase (as measured by SRB staining) in control cells during the drug incubation.

Table 3 provides GI₅₀ values for 5 compounds: compound 4c of Table 1 compound 4a of Table 1, compound 6 of Table 2, compound 20 of Table 2 and compound 28 of Table 2. As can be seen, the compounds tested showed GI₅₀ values in the sub μM to low μM range in general consistent with the data above.

Several of the disclosed compounds were also tested for their ability to inhibit the growth of other cancer cell lines as shown in Tables 4 and 5. The cell lines were obtained from the American Type Culture Collection (Rockville, Md.) and were grown as follows: MCF-7 (p53 wt) breast cancer cells were grown in DMEM media containing 1 mM non-essential amino acids, Earle's BSS, 1 mM Na pyruvate, and 10 mg/L bovine insulin. MDA-MB-468 (p53 mt) cells were grown in DMEM/F-12 Ham's media (1:1 mixture); H838 (p53 wt) cells were grown in RPMI 1640. H358 (p53 null), H1299 (p53 null) cells were grown in RPMI 1640 supplemented with 1.5 g/L Na₂CO₃; 4.5 g/L glucose, 10 mM HEPES buffer, 1 mM Na pyruvate and 2 mM L-glutamine. A549 (p53 wt) cells were grown in Ham's F12K medium supplemented with 2 mM L-glutamine and 1.5 g/L Na₂CO₃; LNCaP (p53 wt) cells were grown in RPMI 1640 supplemented with 1.5 g/L Na₂CO₃, 4.5 g/L glucose, 10 mM HEPES buffer, 1 mM Na pyruvate and 2 mM L-glutamine. A549 (p53 wt) cells were grown in Ham's F12K medium supplemented with 2 mM L-glutamine and 1.5 g/L Na₂CO₃. PC3 (p53 null) cells were cultured in Ham's F-12K medium containing 2 mM L-glutamine. TRAMP Cl cells were cultured in DEME medium with 4 mM L-glutamine adjusted to contain 1.5 g/L sodium bicarbonate and 4.5 g/L glucose supplemented with 0.005 mg/ml bovine insulin and 10 nM dehydroisoandrosterone, 5% fetal bovine serum and 5% Nu-Serum IV; HPAC (p53 wt) pancreatic cancer cells were grown in a 1:1 mixture of DMEM and Ham's F12 medium containing 1.2 g/L Na₂CO₃, 2.5 mM L-glutamine, 15 mM HEPES and 0.5 mM Na pyruvate supplemented with 0.002 mg/mL insulin, 5 μg/mL transferrin, 40 ng/mL hydrocortisone, 10 ng/mL epidermal growth factor and 5% fetal bovine serum. PANC-1 (p53 mt) cells were cultured with RPMI 1640 containing 1 mM HEPES buffer, 25 μg/mL gentamicin, 1.5 g/L Na₂CO₃ and 0.25 μg/mL amphotericin B. BxPC-3 (p53 mt) cells were grown in RPMI 1640 medium, MiaPaCa-2 (p53 mt) and S2013 (p53 mt) cells were grown in DMEM, cell line CFPAC1 (p53 mt) was grown in Iscove's MEM supplemented with 4 mM L-glutamine; T98G (p53 mt) glioma cells were cultured with DMEM supplemented with 1% Na pyruvate, and 1% non-essential amino acids; HCT116 human colon cancer cell lines were kindly provided by Dr. Bert Vogelstein (Johns Hopkins Oncology Center, Baltimore, Md.). The HCT116 cell line was maintained in McCoy's-5A media; Human bronchial epithelial cell line BEAS 2B was cultured in DMEM medium; Human mammary epithelial cell line MCF10A was cultured in a 1:1 mixture of DMEM and Ham's F12 medium containing 5% horse serum, 15 mM hepes buffer, 10 ug/ml insulin, 20 ng/ml EGF, 100 ng/ml choleratoxin and 0.5 ug/ml hydrocortisone; the human primary fibroblast cell lines IMR90-EEA and IMR90-E1A (transformed using the adenoviral oncogene E1A) were gifts from Dr. S. Lee (Harvard University, Boston, Mass.). IMR90 and IMR90-E1A cells were cultured in DMEM medium.

All media contained 10% FBS and 1% penicillin/streptomycin unless otherwise specified. The effects of disclosed compounds on human cancer cell growth, expressed as the percentage of cell survival, were determined by the MTT [3-(4,5-Dimethyl-2-thiazolyl)-2,5-diphenyl-2H-tetrazolium bromide] assay. Cells were grown in 96-well plates and exposed to the test compounds (0, 0.01, 0.1, 1, 10, 100 μM) for 72 hr followed by addition of 10 μL MTT solution (5 mg/mL; Sigma; St. Louis, Mo.) to each well. The plates were incubated for 2-4 hr at 37° C. The supernatant was then removed and formazan crystals were dissolved with 100 μL of DMSO. The absorbance at 570 nm was recorded with an OPTImax microplate reader (Molecular Devices; Sunnyvale, Calif.). The cell survival percentages were calculated by dividing the mean OD of compound containing wells by that of DMSO-control wells. Three separate experiments were accomplished to determine the IC₅₀.

For all cancer type cells, compound 4c of Table 1 and compound 28 of Table 2 had the lowest IC₅₀ values (in nM range) compared with other compounds tested. Compounds 1, 4d and 20 also showed IC₅₀ values in the nM to μM range. Non-cancer cell lines were generally less sensitive to the inhibitory effects of the tested compounds than the cancer cell lines as shown in Table 4. Sixteen additional compounds were evaluated for their effects on MCF-7 cell growth in vitro. IC₅₀ values were calculated as shown in Table 5. Again IC₅₀ value were in the nM range for compounds 16, 24, 26, 8, 2, 21, 30, and 12 and in the μM range for compounds 5, 15, 17, 4e, 7c, 4f and 29.

### Induction of Apoptosis

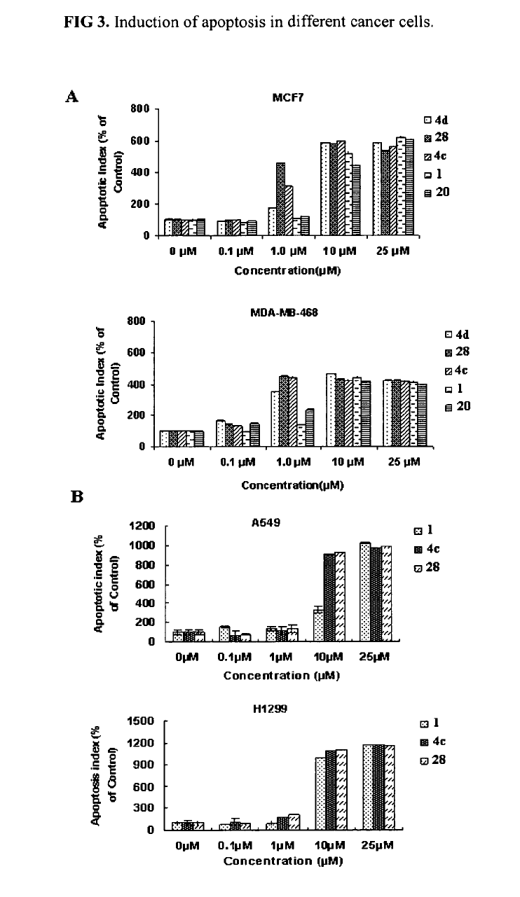

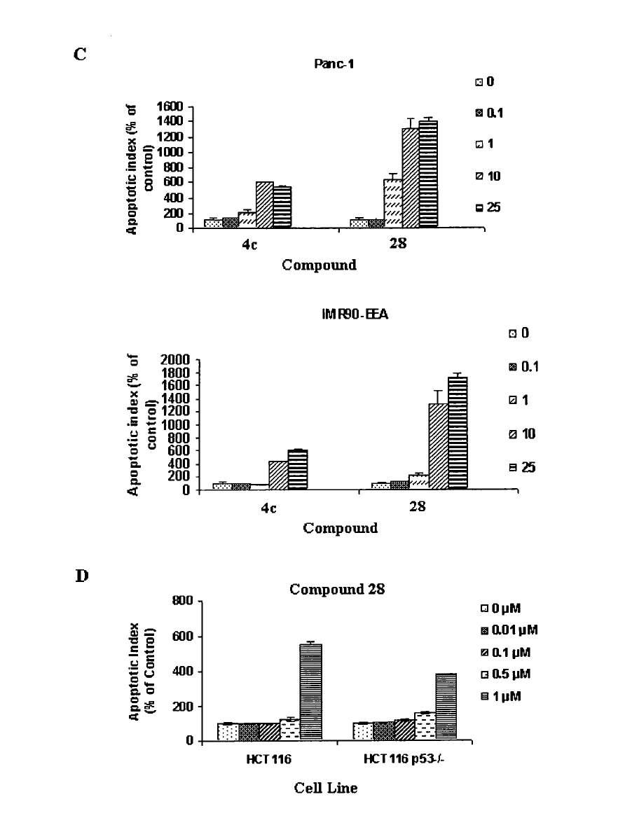

Several of the disclosed compounds were tested for their ability to induce apoptosis in various cell lines as shown in FIG. 3. Cells in early and late stages of apoptosis were detected with an annexin V-FITC apoptosis detection kit from BioVision (Mountain View, Calif.). In brief, cells were exposed to the test compounds (0, 0.1, 1, 10, 25 μM) and incubated for 48 hr prior to analysis. Media and cells were collected and washed with serum-free media. Cells were then re-suspended in 500 μL of Annexin V binding buffer followed by addition of 5 μL Annexin VFITC and 5 μL of propidium iodide (PI). The samples were incubated in the dark for 5 min at room temperature prior to analysis. Samples were analyzed with a Becton Dickinson FACSCalibur instrument (Ex=488 nm; Em=530 nm). Cells that were positive for Annexin V-FITC alone (early apoptosis) and Annexin V-FITC and PI (late apoptosis) were counted. In a dose-dependent manner, these compounds induced apoptosis in different cancer cell lines (FIGS. 3A, B, C, and D). Compound 4c and compound 28 were generally the most effective with significant induction of apoptosis in the 1-10 μM range.

### Inhibition of Cell Cycle Progression

To determination of the effects of the disclosed compounds on the cell cycle, 2-3×10⁵ cells were exposed to the test compounds (0-5 μM) and incubated for 48 hr prior to analysis (Table 6). Cells were trypsinized, washed with PBS and fixed in 1.5 mL 95% ethanol at 4° C. overnight, followed by incubation with RNAse and staining with propidium iodide (Sigma). The DNA content was determined by flow cytometry. For breast cancer (MCF-7), compound 28 induced cell cycle arrest in the S phase in a dose-dependent manner. For lung cancer (A549, H1299), compound 6 induced cell cycle arrest in the G2/M phase in a dose-dependent manner, compound 4c induced cell cycle arrest in the G2/M phase in a dose-dependent manner and compound 28 induced cell cycle arrest in the S phase in a dose-dependent manner. For colon cancer (HCT116 and HCT116 p53^{-/-}), compound 28 induced cell cycle arrest in the S and G2/M phase in a dose-dependent manner.

### Effect on Protein Expression

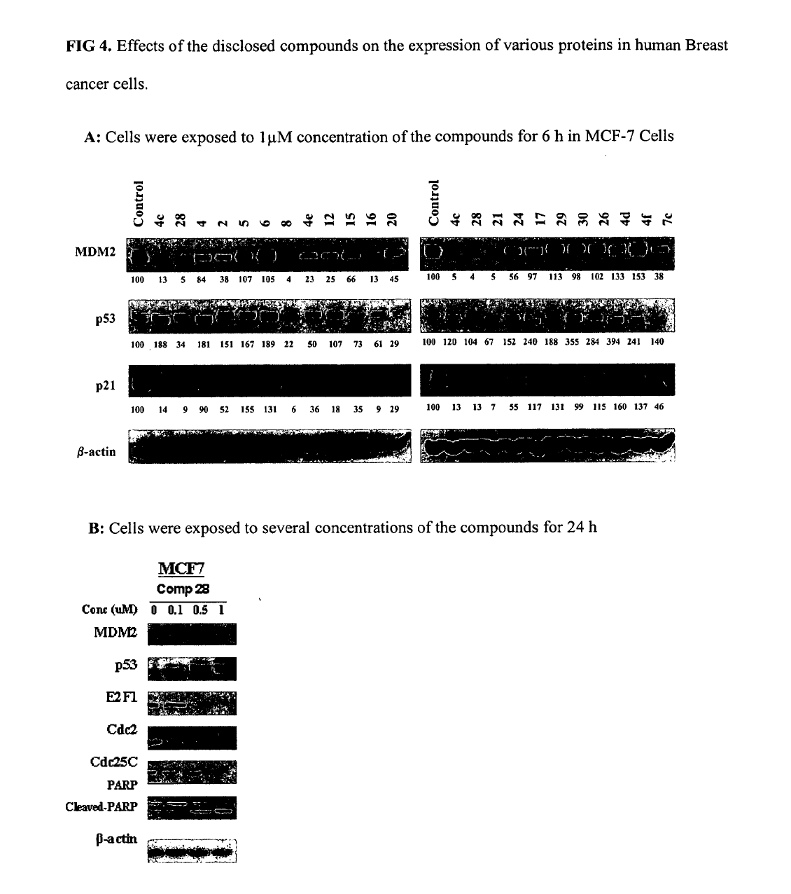

To determine the disclosed compounds effect on protein expression in human breast cancer cells, cultured MCF-7 cells were exposed to various concentrations of the test compounds for 6 h (FIG. 4A) or 24 h (FIG. 4B). Cell lysates were fractionated with identical amounts of protein by SDS-PAGE and transferred to Bio-Rad trans-Blot nitrocellulose membranes (Bio-Rad Laboratories, Hercules, Calif.). The nitrocellulose membrane was incubated in blocking buffer (Tris-buffered saline containing 0.1% Tween 20 and 5% nonfat milk) for 1 h at room temperature, then with the appropriate primary antibody overnight at 4° C. or 2 h at room temperature with gentle shaking. Finally, the membrane was washed three times with the washing buffer (Tris-buffered saline containing 0.1% Tween 20) for 15 min, incubated with goat anti mouse/rabbit IgG-horseradish peroxidase-conjugated antibody (Bio-Rad) for 1 h at room temperature, and washed again (in triplicate). The protein of interest was detected by enhanced chemiluminescence reagents from PerkinElmer LAS, Inc (Boston, Mass.).

Initial screening for the MDM2, p53 and p21 protein levels after treatment with the compounds indicated showed that several compounds inhibited the expression of proteins involved in the regulation of cell growth in MCF-7 cells (FIG. 4A). Compounds 4c, 28, 42, 8, 4e, 12, 15, 16, 20, 21, 24 and 7c inhibited the expression of MDM2; compounds 4d and 4f increased the expression of MDM2. Compounds 4c, 4, 2, 5, 6, 12, 24, 17, 29, 30, 26, 4d, 4f and 7c increased the expression of p53; compounds 28, 8, 4e, 15, 16, 20 and 21 decreased the expression of p53. Compounds 4c, 28, 2, 8, 4e, 12, 15, 16, 20, 21, 24, and 7c decreased the expression of p21; compounds 5, 1, 17, 29, 30, 26, 4d and 4f increased the expression of p21.

Possible mechanism(s) responsible for the pro-apoptotic and cell cycle regulatory effects of compound 28 were further investigated by evaluating its effects on the expression of various proteins involved in these processes. Exposure of cells to compound 28 caused the changes in expression of several cell cycle regulatory proteins. In MCF-7 breast cancer cells (FIG. 4B), the expression levels of MDM2, E2F1, and cdc2, cdc25c were decreased. Furthermore, compound 28 increased expression of cleaved PARP in MCF-7 cells (the increased expression of PARP is indicative of apoptosis.

### Topoisomerase II Assay and Inhibitor Analysis

In order to determine the effect of the makaluvamine derivatives of the present disclosure, the synthesized makaluvamine derivatives were screened for their ability to inhibit topoisomerase II activity in vitro. Topoisomerase II functions by generating a double-stranded DNA break followed by resealing of the break. Topoisomerase II inhibitors interfere with the breakage-rejoining reaction thereby trapping the enzyme in a cleavage complex. In order to determine if the disclosed makaluvamine derivatives inhibited topoisomerase II activity, nine of the derivatives were tested for topoisomerase II inhibitory activity using a topoisomerase-II drug screening kit (TopoGEN, Inc., Port Orange, Fla.) according to the manufacturer's instructions. The screening kit uses a supercoiled plasmid DNA substrate (pRYG) which contains one topoisomerase II cleavage/recognition site. Briefly, topoisomerase II (4 U) was incubated with 500 ng plasmid DNA containing vehicle (DMSO) or 100 μM makaluvamines derivative as described in the protocol supplied with the screening kit. m-AMSA and etoposide were used as positive controls. Relaxed DNA was separated using non-ethidium bromide (EtBr) agarose gels, then stained with EtBr and quantified using Kodak Gel Logic Imaging System and Molecular Imaging software (Eastman Kodak Co., Rochester, N.Y.). Inhibition of relaxation of plasmid DNA or catalytic activity was reported as; − none, + low, ++ moderate, or +++ strong.

The results are summarized in Table 1. Five out of nine tested makaluvamine derivatives (4c, 4d, 4f, 7c and 7e) exhibited inhibition of topoisomerase II catalytic activity comparable to etoposide and m-AMSA. Three of these compounds (4f, 7c and 7e) showed the strongest inhibition of catalytic activity of topoisomerase II.

### In Vivo Administration

To evaluate the potential toxicity of the makaluvamine compounds disclosed, compound 4c was identified for testing in a mouse model. Compound 4c was identified to be a potent inhibitor of HCT-116, MCF-7 and MDA-MB-468 cells, as well as an inhibitor of topoisomerase II. Four groups of 5 female, athymic nude mice (Federick Cancer Research, Rockville, Md.) were injected intra-peritoneally, at doses of 8, 20 or 40 mg/kg of compounds 4c (treatment group) or vehicle only (control group). The 8 mg/kg dose corresponds to the standard etoposide dose given to mice and human patients. As discussed above, etoposide is comparable topoisomerase II inhibitor. The 20 mg/kg and 40 mg/kg groups received 2.5- and 5-times the standard etoposide dose, respectively. Compound 4c was injected at its respective dose in a 200 μl volume of saline:DMSO (100 μl:100 μl). The vehicle only group was injected with 200 μl saline:DMSO. Injections were given 1 time per week for 3 weeks. Mice were observed for 21 days and all surviving mice were sacrificed one week after the last injection. Mice in each groups were evaluated for survival and weight gain/loss during the treatment period.

All mice in the test group and the control group survived (100%) treatment. Furthermore, all mice gained weight in each treatment group. No significant weight changes were observed between each treatment group. These data suggest that the makaluvamine derivatives disclosed herein are not toxic to mammalian subjects. The model used as described herein has shown excellent correlation with in vivo results in humans.

### Pharmaceutical Compositions, Modes of Administration and Methods of Treatment

The present disclosure provides compounds of the general formula (I), (II) as detailed above. The present disclosure also provides for a pharmaceutical composition comprising a therapeutically effective amount of at least one compound of general formula (I) and/or (II). Such pharmaceutical compositions may be used in the manufacture of a medicament for use in the methods of treatment and prevention described herein. Such pharmaceutical compositions and medicaments may comprise a pharmaceutically acceptable carrier, excipients and other additives as known in the art. The compounds of the disclosure are useful in both free form and in the form of pharmaceutically acceptable salts and each may be incorporated into the described pharmaceutical compositions and medicaments.

The pharmaceutically acceptable carriers described herein, including, but not limited to, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds described in the instant disclosure can be administered by any conventional method available for use in conjunction with pharmaceuticals, either as individual therapeutic agents or in combination with additional therapeutic agents.

The compounds described are administered in therapeutically effective amount. The therapeutically effective amount of the compound and the dosage of the pharmaceutical composition administered will, of course, vary depending upon known factors, such as the pharmacodynamic characteristics of the particular agent and its mode and route of administration, the age, health and weight of the recipient; the severity and stage of the disease state or condition; the kind of concurrent treatment; the frequency of treatment; and the effect desired.

A daily dosage of active ingredient can be expected to be about 0.001 to 1000 milligrams (mg) per kilogram (kg) of body weight. In one embodiment, the total amount is between about 0.1 mg/kg and about 1000 mg/kg of body weight; in an alternate embodiment between about 1.1 mg/kg and about 100 mg/kg of body weight; in yet another alternate embodiment between 0.1 mg/kg and about 30 mg/kg of body weight. The above described amounts may be administered as a series of smaller doses over a period of time if desired. As would be obvious, the dosage of active ingredient may be given other than daily if desired.

The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

Dosage forms of the pharmaceutical compositions described herein (forms of the pharmaceutical compositions suitable for administration) contain from about 0.1 mg to about 500 mg of active ingredient (i.e. the compounds disclosed) per unit. In these pharmaceutical compositions, the active ingredient will ordinarily be present in an amount of about 0.5-95% weight based on the total weight of the composition. Multiple dosage forms may be administered as part of a single treatment.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation via the pulmonary system, such as by propellant based metered dose inhalers or dry powders inhalation devices. Other dosage forms are potentially possible such as administration transdermally, via patch mechanism or ointment.

Formulations suitable for oral administration can consist of (a) liquid solutions, such as a therapeutically effective amount of the compound dissolved in diluents, such as water, saline, or orange juice; (b) capsules, sachets, tablets, lozenges, and troches, each containing a predetermined Therapeutically effective amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound can be administered in a physiologically acceptable diluent in a pharmaceutically acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl)-3-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The compounds of the present disclosure, alone or in combination with other suitable components, can be made into aerosol formulations to be administered via inhalation. These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutically acceptable carriers for injectable compositions are well known to those of ordinary skill in the art. See Pharmaceutics and Pharmacy Practice, J. B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include pastilles comprising the active ingredient in an inert base, such as gelatin and glycerin, or sucrose and acacia, as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

One skilled in the art will appreciate that suitable methods of administering a compound of the present invention to an patient are available, and, although more than one route can be used to administer a particular compound, a particular route can provide a more immediate and more effective reaction than another route.

The foregoing description illustrates and describes the compounds of the present disclosure. Additionally, the disclosure shows and describes only the preferred embodiments of the compounds but, as mentioned above, it is to be understood that the teachings of the present disclosure are capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. The claim below is appended for purposes of foreign priority only, if required. All references cited herein are incorporated by reference as if fully set forth in this disclosure.

TABLE 1
In vitro cytotoxicity and inhibition of topoisomerase II catalytic activities of
makaluvamine derivatives disclosed herein.

| | | | Inhibition of Cancer Cell | | | |
|---|---|---|---|---|---|---|
| | | | Proliferation (IC₅₀, ^{μ}M)^{a} | | | Topoisom- |
| | Cpd | | | | MDA-MB- | erase II |
| Structure | No | R = | HCT-116 | MCF-7 | 468 | Inhibition^{b} |
| L = | Compound I | | | | | |
| L = 0 | 4a | —CH₃ | 2.5 ± 1.3 | 1.3 ± 0.1 | 0.4 ± 0.05 | — |
| L = CH₂ | 4b | —CH₃ | 3.6 ± 0.7 | 1.3 ± 0.3 | 0.4 ± 0.03 | — |
| L = CH₂ | 4c | 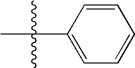 | 1.3 ± 0.2 | 1.0 ± 0.4 | 0.3 ± 0.18 | ++ |
| L = CH₂CH₂ | 4d | 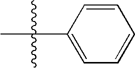 | 3.9 ± 1.2 | 1.7 ± 0.5 | 0.3 ± 0.27 | + |
| L = CH₂CH₂ | 4e | 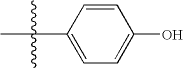 | 13.2 ± 0.9 | 3.2 ± 0.6 | 0.6± 0.05 | — |
| L = CH₂CH₂ | 4f | 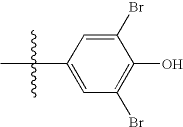 | 14.4 ± 1.2 | 13.2 ± 3.1 | 4.5 ± 0.8 | +++ |
| L = CH₂CH₂ | 4g | 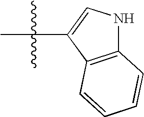 | 5.2 ± 0.8 | 1.6 ± 0.2 | 0.4 ± 0.01 | — |
| L = | Compound II | | | | | |
| L = CH₂ | 7c | 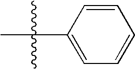 | 2.7 ± 1.5 | 1.8 ± 0.2 | 1.0 ± 0.01 | +++ |
| L = CH₂CH₂ | 7d | 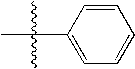 | 0.5 ± 0.03 | 1.5 ± 0.1 | 0.8 ± 0.1 | —^{d} |
| L = CH₂CH₂ | 7e | 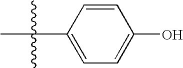 | 5.3 ± 0.6 | 5.1 ± 0.4 | 1.5 ± 0.14 | +++ |
| L = CH₂CH₂ | 7f | 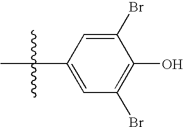 | 1.0 ± 0.2 | 2.3 ± 1.1 | 1.5 ± 0.9 | —^{d} |
| L = CH₂CH₂ | 7g | 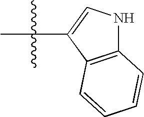 | 0.8 ± 0.3 | 1.2 ± 0.03 | 1.4 ± 0.9 | —^{d} |

| | | | | | |
|---|---|---|---|---|---|
| Etoposide | —^{c} | 1.7 ± 0.2 | 35.6 ± 3.4 | 13.6 ± 0.6 | + |
| m-AMSA | —^{c} | 0.7 ± 0.3 | 21.7 ± 2.5 | 8.5 ± 1.2 | ++ |

^{a}The dose that inhibits 50% cell proliferation (IC₅₀) was determined in human colon cancer cell line, HCT-116, and the human breast cancer cell lines, MCF-7 and MDA-MB-468 (ATCC, Manassas, VA). The IC₅₀ dose from 2-4 independent XTT assays performed in triplicate were combined for an average ± standard deviation.
^{b}Topoisomerase II (4U) was incubated with 500 ng plasmid DNA containing vehicle (DMSO) or 100 μM drug compound as described in the Topo II Drug Screening Kit protocol (TopoGEN, Inc). m-AMSA and etoposide were used as positive controls. Relaxed DNA was separated using non-ethidium bromide (EtBr) agarose gels, then stained with EtBr and quantified using Kodak Gel Logic Imaging System and Molecular Imaging software (Eastman Kodak Co., Rochester, NY). Inhibition of relaxation of plasmid DNA or catalytic activity was reported as; — none, + low, ++ moderate, or +++ strong.
^{c}not applicable;
^{d}not determined.

TABLE 2
IC₅₀ of several of the makaluvamines disclosed herein in MCF-7 cells.

| | | | IC₅₀ (μM) | |
|---|---|---|---|---|
| No | MW | Structure | MCF-7 | |
| 1 | 589 | 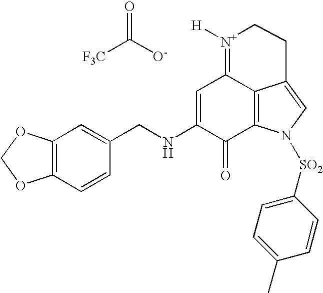 | 0.78 | μM |
| 2 | 435 | 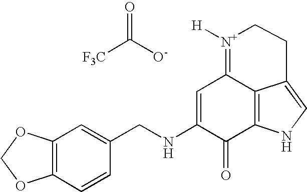 | 5.22 | μM |
| 3 | 635 | 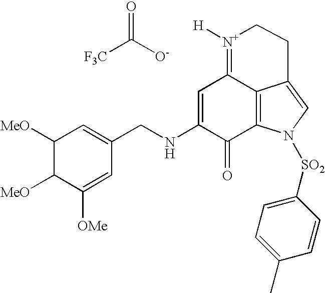 | 0.96 | μM |
| 4 | 481 | 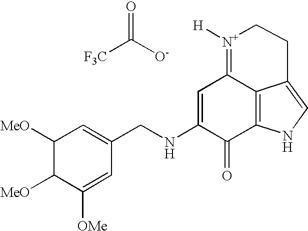 | 30.50 | μM |
| 5 | 603 | 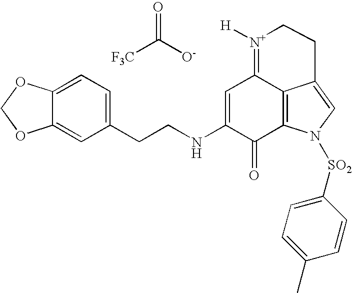 | —^{a} | |
| 6 | 449 | 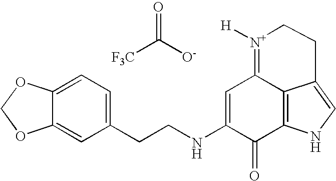 | 2.44 | μM |
| 7 | 604 | 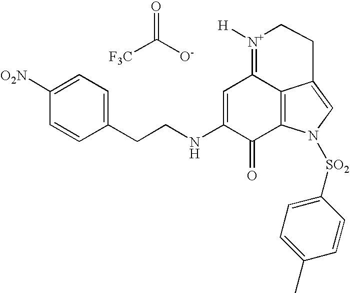 | >1 | μM |
| 8 | 450 | 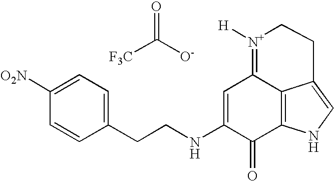 | 2.82 | μM |
| 9 | 559 | 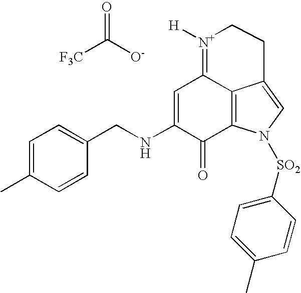 | 1.48 | μM |
| 10 | 405 | 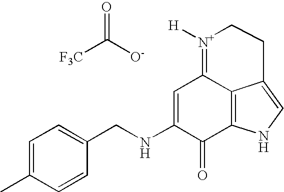 | 1.69 | μM |
| 11 | 573 | 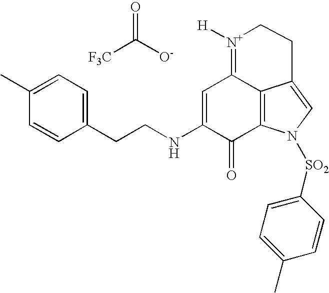 | 1.47 | μM |
| 12 | 419 | 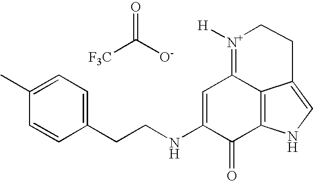 | 2.84 | μM |
| 13 | 575 | 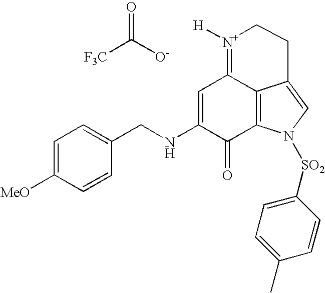 | 1.02 | μM |
| 14 | 421 | 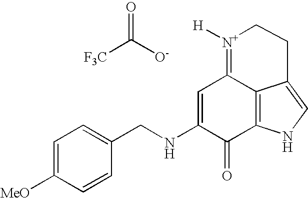 | 2.68 | μM |
| 15 | 589 | 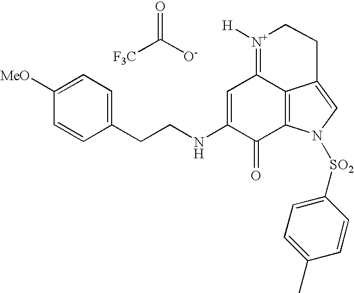 | 1.33 | μM |
| 16 | 435 | 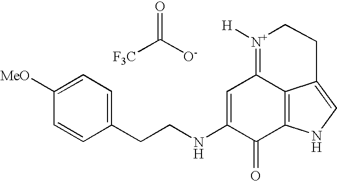 | 2.57 | μM |
| 17 | 605 | 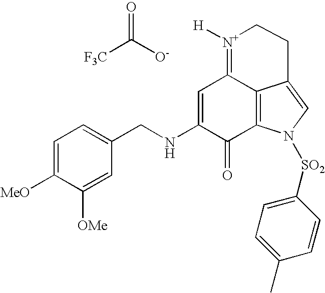 | 0.65 | μM |
| 18 | 451 | 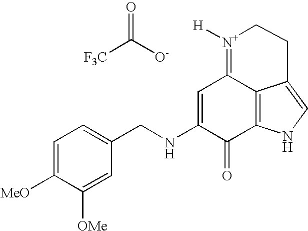 | 5.98 | μM |
| 19 | 619 | 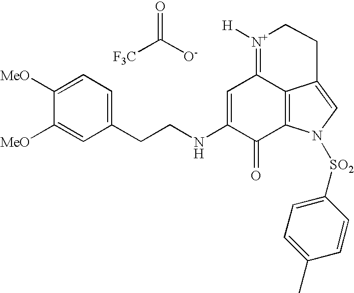 | >1 | μM |
| 20 | 465 | 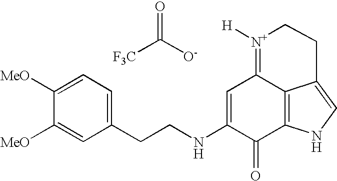 | 10.90 | μM |
| 21 | 579 | 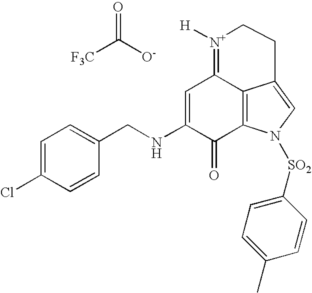 | 0.83 | μM |
| 22 | 425 | 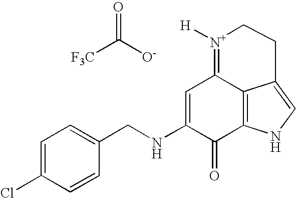 | 1.33 | μM |
| 23 | 593 | 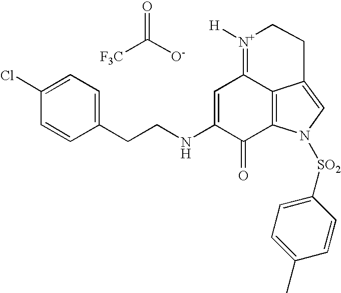 | 1.55 | μM |

| | | | |
|---|---|---|---|
| 24 | 439 | 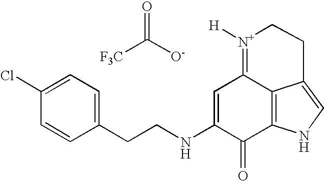 | —^{a} |
| 25 | 628 | 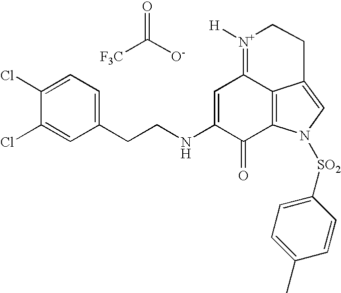 | —^{a} |
| 26 | 474 | 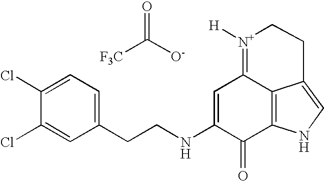 | —^{a} |
| 27 | 563 | 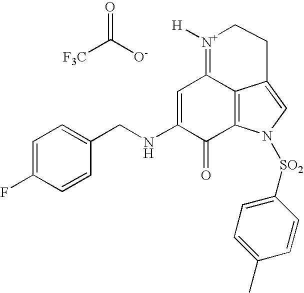 | —^{a} |
| 28 | 409 | 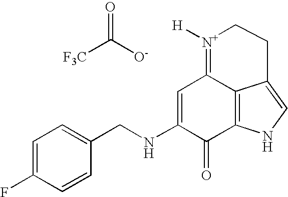 | —^{a} |
| 29 | 577 | 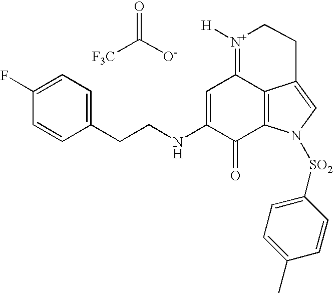 | —^{a} |
| 30 | 423 | 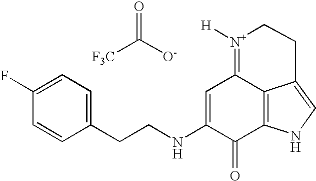 | —^{a} |

| | | |
|---|---|---|
| Etoposide control | 30.5 | μM |

TABLE 3
Results of the NCI screen.

| Cell Line | 4c | 6 | 4a | 28 | 20 |
|---|---|---|---|---|---|
| Leukemia | | | | | |
| CCRF-CEM | 0.900 | 2.97 | 2.97 | 1.43 | 10.6 |
| HL-60 | 0.325 | 1.92 | 2.54 | 1.53 | 3.14 |
| K-562 | 2.36 | 3.43 | 2.19 | 1.07 | 16.9 |
| MOLT-4 | 0.315 | 2.02 | 1.97 | 0.793 | 4.95 |
| RPMO-8226 | 1.36 | n.d. | 5.99 | 0.559 | n.d. |
| SR | 1.75 | 2.50 | 3.12 | 1.21 | 5.69 |
| Non-Small Cell | | | | | |
| Lung Cancer | | | | | |
| A549/ATCC | 2.14 | n.d. | 2.78 | 1.44 | 2.72 |
| EKVX | 0.271 | 1.96 | 2.20 | 0.453 | 3.08 |
| HOP-62 | 3.70 | 2.50 | 1.28 | 3.79 | 5.17 |
| HOP-92 | 0.308 | 5.45 | 1.28 | 1.68 | 1.03 |
| NCI-H226 | 5.45 | 4.38 | 11.1 | 2.34 | 1.06 |
| NCI-H23 | 1.47 | 1.69 | 2.46 | 0.521 | 2.00 |
| NCIH332M | 0.628 | 2.82 | 2.89 | 1.17 | 2.84 |
| NCI-H460 | 1.51 | 1.01 | 2.16 | 0.816 | 1.42 |
| NCI-H552 | 1.69 | 2.62 | 4.13 | 2.00 | 2.24 |
| Colon Cancer | | | | | |
| COLO 205 | 0.378 | 1.02 | 1.75 | 0.365 | 1.39 |
| HCC-2998 | 2.61 | 1.56 | 4.91 | 2.70 | 2.35 |
| HCT-116 | 0.306 | 2.41 | 2.94 | 0.715 | 2.40 |
| HCT-15 | 1.39 | 1.86 | 5.09 | 1.48 | 2.65 |
| HT-29 | 0.488 | 2.07 | 2.54 | 0.596 | 2.54 |
| KM12 | 2.06 | 2.29 | 3.83 | 2.02 | 2.23 |
| SW-620 | 1.07 | 3.44 | 3.10 | 1.82 | 3.09 |
| CNS Cancer | | | | | |
| SF-268 | 2.83 | 3.66 | 2.09 | 1.66 | <0.010 |
| SF-295 | 1.12 | 1.87 | 1.21 | 1.06 | 1.84 |
| SF-539 | 1.91 | 2.21 | 2.39 | 1.74 | 3.28 |
| SNB-19 | 1.96 | 3.29 | 3.43 | 1.79 | 3.26 |
| SNB-75 | 1.65 | 3.49 | 1.38 | 5.20 | 5.22 |
| U251 | 2.66 | 3.05 | 4.20 | 1.99 | 3.44 |
| Melanoma | | | | | |
| LOX IMVI | 1.81 | 1.68 | 3.43 | 1.57 | 2.34 |
| MALME-3M | 0.300 | 0.343 | 1.21 | 0.208 | 0.249 |
| M14 | 1.28 | 1.43 | 2.57 | 1.44 | 2.26 |
| SK-MEL-2 | 5.20 | 5.82 | 1.67 | 4.53 | 0.730 |
| SK-MEL-28 | 7.42 | 4.91 | 6.62 | 5.78 | 10.1 |
| SK-MEL-5 | 0.462 | 0.965 | 1.45 | 0.325 | 1.33 |
| UACC-257 | 0.315 | 2.97 | 2.00 | 0.189 | 3.07 |
| UACC-62 | 1.82 | 2.32 | 2.49 | 1.56 | 2.17 |
| Ovarian Cancer | | | | | |
| IGROV1 | 0.793 | 2.52 | 2.90 | 1.43 | 4.24 |
| OVCAR3 | 2.58 | 2.02 | 6.30 | 2.23 | 1.61 |
| OVCAR4 | 0.624 | 0.971 | 1.08 | 1.09 | 1.16 |
| OVCAR5 | 2.67 | 2.72 | 17.3 | 2.37 | 5.14 |
| OVCAR8 | 2.08 | 1.62 | 3.19 | 1.97 | 1.79 |
| SK-OV-3 | 7.01 | 9.70 | 18.7 | 3.15 | 14.6 |
| Renal Cancer | | | | | |
| 786-0 | 3.22 | 3.98 | 10.5 | 3.45 | 5.43 |
| A498 | 16.5 | 6.03 | 20.7 | 3.11 | 7.24 |
| ACHN | 2.8 | 3.25 | 13.1 | 2.04 | 4.21 |
| CAKI-1 | 0.298 | 1.77 | 1.95 | 0.329 | 2.67 |
| RXF 393 | 2.09 | n.d. | 4.22 | 0.010 | n.d. |
| SN12C | 1.89 | 2.69 | 3.06 | 1.95 | 3.07 |
| TK-10 | 4.72 | 4.51 | 15.4 | 3.19 | 5.10 |
| UO-31 | 2.63 | 5.84 | 14.6 | 2.59 | 7.11 |
| Prostate Cancer | | | | | |
| PC-3 | 1.54 | 5.93 | 3.61 | 1.57 | 7.62 |
| DU-145 | 1.79 | 1.35 | 2.29 | 1.08 | 2.81 |
| Breast Cancer | | | | | |
| MCF-7 | 0.061 | 1.21 | 1.52 | 0.160 | 2.61 |
| NCI/ADR-RES | 3.51 | 1.76 | 9.33 | 3.11 | 12.4 |
| MDA-MB- | 0.439 | 2.32 | 3.13 | 0.725 | 3.21 |
| 231/ATCC | | | | | |
| HS 578T | 5.20 | 2.86 | 10.2 | 44.0 | 5.04 |
| MDA-MB-435 | 2.03 | 1.89 | 1.80 | 1.75 | 2.05 |
| BT-459 | 4.58 | 3.07 | 7.38 | 3.14 | 4.53 |
| T-47D | 0.523 | 2.97 | 3.50 | 0.633 | 2.49 |

*The above values are GI₅₀ values illustrated in μM

TABLE 4
Growth inhibitory activity (IC₅₀) of the disclosed compounds
in various cell lines (IC₅₀ values shown in μM).

| | 4d | 28 | 4c | 6 | 20 |
|---|---|---|---|---|---|
| Cancer Cell | | | | | |
| MW | 405 | 409 | 391 | 449 | 465 |
| MDA-MB-468 | 0.101 | 0.125 | 0.126 | 0.428 | 0.277 |
| LNCaP | 0.161 | 1.290 | 2.754 | 3.959 | 24.451 |
| H358 | 0.398 | 0.170 | 0.436 | 1.204 | 0.983 |
| H838 | 0.260 | 0.110 | 0.160 | 0.665 | 0.838 |
| HPAC | 1.131 | 0.535 | 0.689 | 2.920 | 2.852 |
| Panc-1 | 0.258 | 0.104 | 0.141 | 0.587 | 0.720 |
| Mia-paca | 0.982 | 0.315 | 0.345 | 2.740 | 1.320 |
| H1299 | 4.268 | 0.968 | 1.417 | 5.945 | 5.989 |
| U87MG | 4.276 | 1.707 | 2.876 | 5.643 | 3.037 |
| S2013 | 5.391 | 4.453 | 3.853 | 11.205 | 29.627 |
| CFPAC1 | 5.114 | 5.398 | 8.768 | 9.098 | 4.273 |
| BXPC3 | 2.941 | 2.297 | 2.874 | 3.211 | 5.014 |
| Normal Cells | | | | | |
| IMR90-EEA | 3.774 | 1.488 | 2.772 | 5.698 | 2.880 |
| MCF10A | 6.815 | 5.332 | 5.269 | 13.110 | 3.397 |
| BEAS-2B | 0.695 | 0.587 | 1.445 | 2.239 | 1.687 |

TABLE 5
Growth inhibitory activity (IC⁵⁰) of the 16
of the disclosed compounds in MCF7 cells.

| | 16 | 24 | 26 | 8 |
|---|---|---|---|---|
| MW | 435 | 439 | 474 | 450 |
| IC50 | 0.200 | 0.584 | 0.495 | 0.427 |
| | 2 | 4 | 5 | 15 |
| MW | 435 | 481 | 603 | 589 |
| IC50 | 0.809 | 0.515 | 1.619 | 1.049 |
| | 21 | 17 | 4e | 7c |
| MW | 579 | 605 | 421 | 545 |
| IC50 | 0.774 | 1.009 | 1.071 | 1.205 |
| | 4f | 30 | 29 | 12 |
| MW | 579 | 423 | 577 | 419 |
| IC50 | 2.722 | 0.787 | 1.919 | 0.833 |

TABLE 6
Effects of the disclosed compounds on the cell
cycle progression of different cancer cells.

| | G1 | S | G2/M |
|---|---|---|---|
| MCF7 Compound 28 | | | |
| 0 μM | 80.24 | 7.09 | 12.68 |
| 0.1 μM | 75.10 | 7.37 | 17.53 |
| 0.5 μM | 51.97 | 27.86 | 20.17 |
| 1 μM | 53.96 | 29.82 | 16.21 |
| HCT116 Compound 28 | | | |
| 0 μM | 58.75 | 14.87 | 26.38 |
| 0.5 μM | 31.82 | 23.14 | 45.04 |
| 1 μM | 28.40 | 33.43 | 38.17 |
| HCT-116 p53 −/−− Compound 28 | | | |
| 0 μM | 50.32 | 17.99 | 31.69 |
| 0.5 μM | 28.18 | 22.80 | 49.02 |
| 1 μM | 25.59 | 29.21 | 45.20 |
| A549 Compound 28 | | | |
| 0 μM | 59.63 | 20.10 | 20.27 |
| 0.1 μM | 59.91 | 19.87 | 20.22 |
| 1 μM | 57.88 | 21.98 | 20.14 |
| 5 μM | 46.78 | 28.14 | 25.07 |
| H1299 Compound 28 | | | |
| 0 μM | 54.27 | 20.16 | 25.58 |
| 0.1 μM | 54.23 | 20.60 | 25.17 |
| 1 μM | 46.88 | 21.31 | 31.82 |
| 2 μM | 31.52 | 32.11 | 36.36 |
| A549 Compound 6 | | | |
| 0 μM | 60.18 | 19.48 | 20.34 |
| 0.1 μM | 58.66 | 20.50 | 20.85 |
| 1 μM | 58.59 | 21.04 | 20.37 |
| 5 μM | 50.88 | 25.76 | 23.36 |
| A549 Compound 4c | | | |
| 0 μM | 59.63 | 20.10 | 20.27 |
| 0.1 μM | 60.23 | 20.37 | 19.41 |
| 1 μM | 58.62 | 21.53 | 19.85 |
| 5 μM | 47.18 | 22.48 | 30.35 |
| H1299 Compound 6 | | | |
| 0 μM | 54.27 | 20.16 | 25.58 |
| 0.1 μM | 53.99 | 15.28 | 30.73 |
| 1 μM | 57.20 | 15.22 | 27.57 |
| 5 μM | 35.29 | 23.43 | 41.28 |
| H1299 Compound 4c | | | |
| 0 μM | 54.27 | 20.16 | 25.58 |
| 0.1 μM | 55.27 | 17.84 | 26.89 |
| 1 μM | 51.18 | 19.47 | 29.35 |
| 2 μM | 30.46 | 19.96 | 49.58 |

1. A compound having the following structural formula 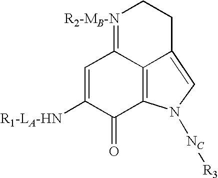
wherein
R₁ is selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;
R₂ and R₃ are each independently selected from H or substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;
L is selected from a substituted or unsubstituted alkyl of 2-15 carbon atoms in length or a substituted or unsubstituted alkynyl of 2-15 carbon atoms in length;
M and N are each independently selected from a substituted or unsubstituted alkyl, substituted or unsubstituted alkenyl, substituted or unsubstituted alkynyl;
A=1;
B and C are each independently selected from 0 or 1, or a pharmaceutically acceptable salt thereof; and
R₁ is selected from:

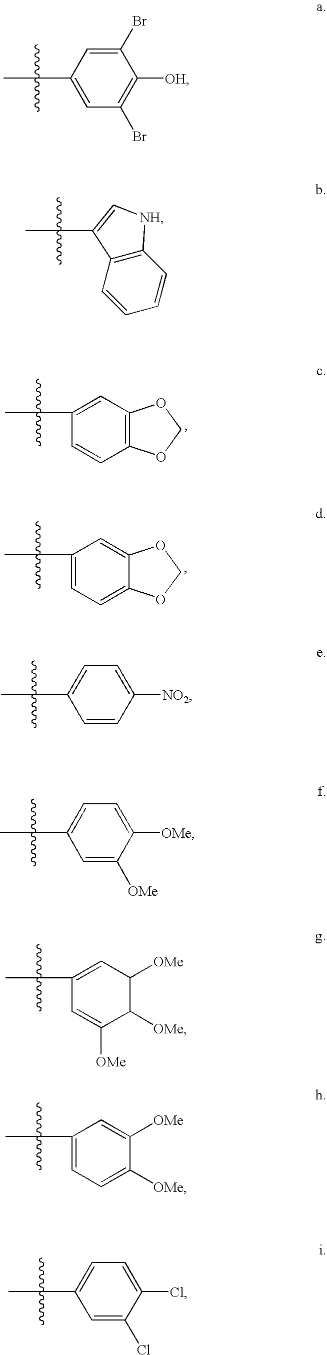

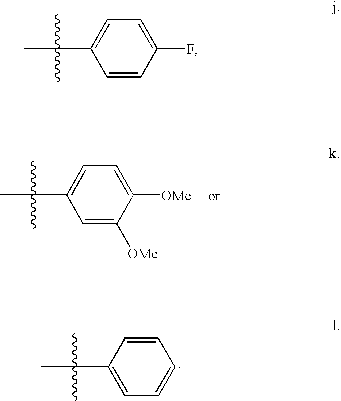

2. (canceled)

3. The compound of claim 1 where B and C are =0 and R₂ and R₃ are H.

4. The compound of claim 1 where B and C are equal to 1 and R₂ and R₃ are each independently selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl.

5. The compound of claim 1 where when B=0, R₂ is H and when C=0, R₃ is H.

6. The compound of claim 1 where L is selected from a substituted or unsubstituted alkyl of 2-15 carbon atoms in length.

7. A compound having the following structural formula 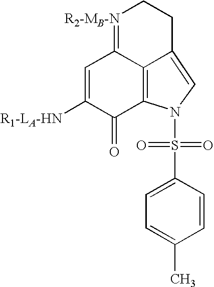
wherein
R₁ is selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;
R₂ is selected from H or substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;
L and M are each independently selected from a substituted or unsubstituted alkyl, substituted or unsubstituted alkenyl, substituted or unsubstituted alkynyl;
A=1; and
B is selected from 0 or 1, or a pharmaceutically acceptable salt thereof.

8. The compound of claim 7 where when B=0, R₂ is H.

9. The compound of claim 7 wherein R₁ is selected from the group consisting of:

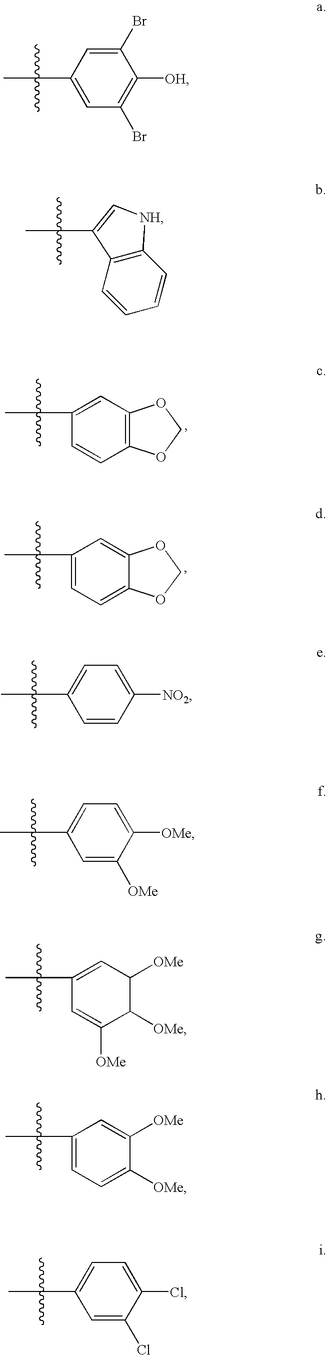

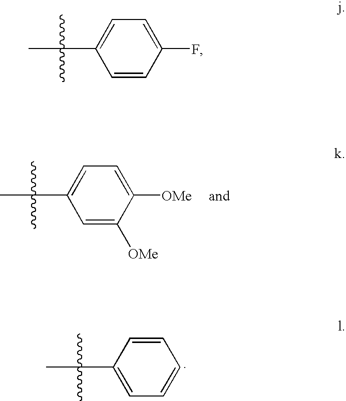

10. The compound of claim 9 where B=0 and R₂═H.

11. The compound of claim 9 where B=to 1 and R₂ is selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl.

12. The compound of claim 7 where L is selected from a substituted or unsubstituted alkyl of 2-15 carbon atoms in length, a substituted or unsubstituted alkenyl of 2-15 carbon atoms in length or a substituted or unsubstituted alkynyl of 2-15 carbon atoms in length.

13. A compound having the following structural formula 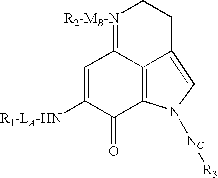
Wherein
R₁ is selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;
R₂ and R₃ are each independently selected from H or substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl;
L, M and N are each independently selected from a substituted or unsubstituted alkyl, substituted or unsubstituted alkenyl, substituted or unsubstituted alkynyl;
A=1; and
B and C are each independently selected from 0 or 1, or a pharmaceutically acceptable salt thereof;
provided that when R₁ is selected from straight chain alkyl of 1-10 carbon atoms, furan, thiopene, napthyl, pyridyl, phenyl, mono-substituted phenyl or di-substituted phenyl (where the substitutions include the following: a straight chain alkyl of 1-4 carbon atoms, trifluoromethyl, nitro, an O-alkyl of 1-4 carbon atoms, hydroxyl, F, Cl, Br, NRₓRₓ, where Rₓ is a straight chain alkyl from 1-4 carbon atoms, or CO₂Rₓ), 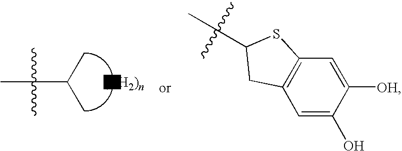
L is not CH₂;
and further provided that when L is a substituted or unsubstituted alkyl or alkenyl of 2 carbon atoms in length, the R₁ is not 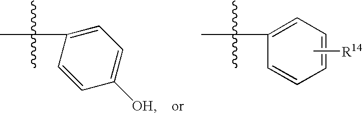
where R₁₄ is F, Cl, Br, NO₂, straight chain alkyl of 1 to 4 carbons, O-alkyl of 1 to 4 carbons or trifluoromethyl.

14. The compound of claim 13 wherein R₁ is selected from:

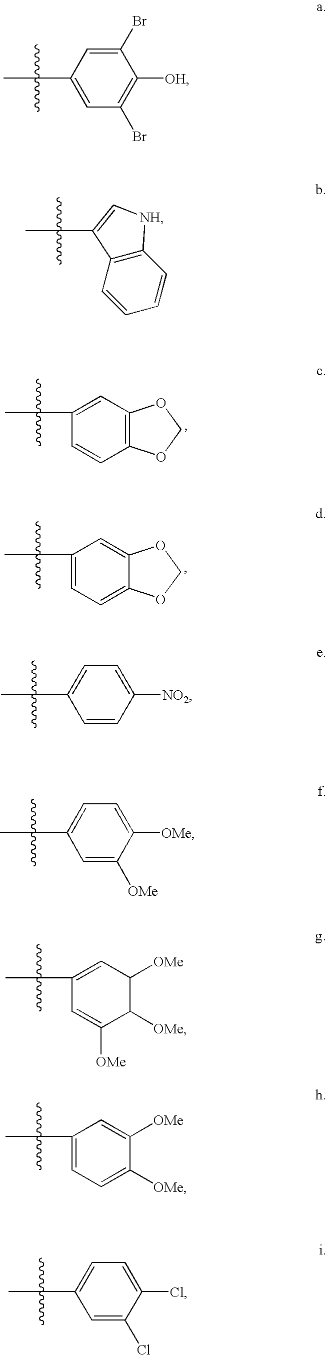

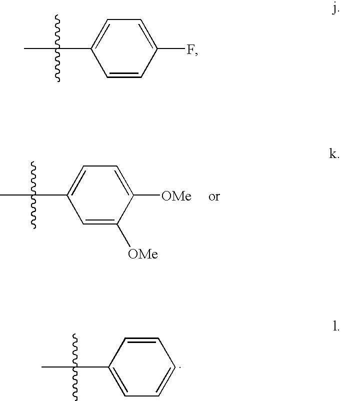

15. The compound of claim 14 where B and C are =0 and R₂ and R₃ are H.

16. The compound of claim 14 where B and C are equal to 1 and R₂ and R₃ are each independently selected from substituted or unsubstituted alkyl, substituted or unsubstituted alkenyl, substituted or unsubstituted alkynyl, substituted or unsubstituted aryl, substituted or unsubstituted heterocyclyl, substituted or unsubstituted aralkyl, or substituted or unsubstituted heterocyclylalkyl.

17. The compound of claim 13 where when B=0, R₂ is H and when C=0, R₃ is H.

18. The compound of claim 13 where L is selected from a substituted or unsubstituted alkyl of 2-15 carbon atoms in length, substituted or unsubstituted alkenyl of 2-15 carbon atoms in length, substituted or unsubstituted alkynyl of 2-15 carbon atoms in length.

19. A method of treating or preventing cancer or a condition characterized by unregulated cellular proliferation in a subject, the method comprising the steps of administering a compound of claim 1, 7, 9 or 13 or 14 to the subject.

20. (canceled)

21. The method of claim 20 wherein the expression, activity or both the expression and activity of at least one of MDM2, E2F1, Cdc2, cdc25c and/or p21 is decreased, the expression, activity or both the expression and activity of p53 is increased or a combination of the foregoing.

22. (canceled)

23. The method of claim 19 wherein the administration induces cell death or cell-cycle arrest.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. A method of treating or preventing a condition characterized by increased MDM2 activity, increased MDM2 expression, decreased p53 activity, increased p53 expression or a combination of the foregoing in a subject, the method comprising the steps of administering a compound of claim 1, 7, 9 13 o4 14 to the subject.

44. (canceled)

45. The method of claim 44 wherein the expression, activity or both the expression and activity of at least one of MDM2, E2F1, Cdc2, cdc25c and/or p21 is decreased, the expression, activity or both the expression and activity of p53 is increased or a combination of the foregoing.

46. (canceled)

47. The method of claim 43 wherein the administration induces cell death or cell-cycle arrest.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. A method of treating or preventing a condition caused by or related to a bacterial infection in a subject, the method comprising the steps of administering a compound of claim 1, 7 9 13 o4 14 to the subject.
